(19) United States
(12) Patent Application Publication
(10) Pub. No.: US 2007/0292486 A1
(43) Pub. Date: Dec. 20, 2007

(54) NOVEL POLYMER-NANO/MICROPARTICLE COMPOSITES

(75) Inventors: Ayusman Sen, State College, PA (US); Varun Sambhy, State College, PA (US)

Correspondence Address: GIFFORD, KRASS, SPRINKLE,ANDERSON & CITKOWSKI, P.C, PO BOX 7021, TROY, MI, 48007-7021, US

(73) Assignee: The Penn State Research Foundation, University Park, PA (US)

(21) Appl. No.: 11/763,141

(22) Filed: Jun. 14, 2007

Related U.S. Application Data
(60) Provisional application No. 60/814,090, filed on Jun. 15, 2006.

(51) Int. Cl. A61K 9/70; A61K 9/14; A61K 33/38; B32B 27/04

(52) U.S. Cl. 424/443; 424/486; 424/618; 442/123

(57) ABSTRACT

Polymeric materials, including polymers and polymer-composite materials, are useful for a variety of applications including biocidal coatings. Representative examples include a composite of silver salt particles and an ionic polymer. A novel process was developed whereby the counter-ion of an ionic polymer is precipitated as a metal salt, so as to form metal salt nanoparticles or microparticles within a polymer matrix. In other examples, polymers having cross-linkable silicon-containing groups form stable biocidal coatings on various substrates, including textiles. Biocidal activity may arise from silver salt particles (or other biocidal particles), ions (such as biocidal anions), membrane disruption by charged species, or some combination thereof. Further, bromide ions, such as provided by silver bromide particles, may impart fire retardant properties to textile substrates.

### REFERENCE TO RELATED APPLICATION

This application claims priority from U.S. Provisional Patent Application Ser. No. 60/814,090, filed Jun. 15, 2006, the entire content of which is incorporated herein by reference.

### FIELD OF THE INVENTION

The invention relates to polymers and polymer-particle composites, including biocidal materials having antibacterial, antifungal, an/or antiviral properties.

### BACKGROUND OF THE INVENTION

Bacterial, fungal, and viral proliferation is a serious problem in many situations, and there are numerous applications for antibacterial surfaces. Hence, improved biocidal surfaces, such as antibacterial surfaces, would find many uses.

Polymer-particle composites find a wide array of applications. Hence, improved synthetic methods to prepare such composites and the composites formed by such improved methods have many uses, including biocidal surfaces having antibacterial, antiviral and/or antifungal properties.

### SUMMARY OF THE INVENTION

Embodiments of the present invention include particle-polymer composites having biocidal properties. Biocidal materials include polymer-particle composites and polymeric materials including biocidal counterions, which may be formed as coatings formed on substrates, such as glass, plastics, metals, oxide surfaces, ceramics, wood, fibers, gels, resins, paints, and textiles.

In some examples, the particles are microparticles or nanoparticles comprising a silver compound, in particular a silver salt such as silver bromide. The polymer may be an ionic polymer, such as a cationic polymer or anionic polymer. Sparingly soluble silver salts release silver ions over extended periods, such as hours or days, providing persistent biocidal properties. Composite materials are described that are effective against a wide range of pathogens, including bacteria, fungi, and viruses. Example biocidal composites and polymers according to the present invention were shown to impede growth of bacteria and fungi. Visibly discernable zones of inhibition were observed around patterned biocidal composite coatings formed on a substrate.

Polymer molecules may include ternary and/or quaternary nitrogen atoms. Representative examples include derivatives of poly(4-vinyl pyridine), including copolymers thereof, such as a copolymer of poly(4-vinyl pyridine) and an N-substituted poly(4-vinyl pyridine), more particularly a copolymer of an N-alkyl substituted poly(4-vinyl pyridine).

In some examples, a polymer includes a cross-linkable and/or surface-binding functional group. Each polymer molecule may include a plurality of such groups, so that a polymer proximate to the surface may form multiple covalent bonds with the surface. The multiple covalent bonds between the polymer and the surface increases the resistance of the coating to removal, for example by solvents or other cleaning processes. For example, a polymer including silane groups may form a plurality of Si—O-(surface atom) bonds to hydroxyl-terminated surfaces. In some examples, a cross-linkable silicon-containing group (such as alkoxysilane groups, halosilane, or other silane group) is linked to the nitrogen atom through a linking group. The linking group may be alkyl, or other carbon-containing chain.

The polymer component of a polymer-particle composite may be cross-linked, for example to increase the robustness of the coating. The cross-linking may use a condensation reaction between hydrolyzable silicon-containing groups (such as alkoxysilane groups, halosilane, or other silane groups) on adjacent polymer chains. Other cross-linking mechanisms may be used. A functional group taking part in the cross-linking process may further act as a surface binding group. In some examples, the cross-linking step to form Si—O—Si crosslinks may occur in the temperature range 25° C.-80° C., allowing low temperature processing if desired. However, higher temperatures may be used, for example for more rapid cross-linking or drying steps. In some examples, a polymeric coating may be covalently bound to the substrate, and example polymers may be capable of melting numerous covalent bonds to the substrate. In other examples, a polymer may be cross-linked without covalently binding to a surface. A biocidal composite coating may comprise a multilayer coating, including at least one layer cross-linked by silicon containing groups. A coating may comprise of one or more cross-linked polymer layers attached to surface by covalent bonds through silicon containing groups.

Example polymers may further include fluorinated groups, such as fluoroalkane groups, and polymers and composites thereof may be hydrophobic. Examples also include ionic polymers having biocidal counterions, such as iodide, triiodide, hypochlorite, other oxidizing anion, and the like.

A process of forming a composite material comprises providing a solution of an ionic polymer, the polymer molecules including charged atomic species and having a counter ion. The polymer solution is treated with a metal compound reagent so that the metal compound reacts with the counter ion to form a metal salt of the counter ion. On forming a film from the solution, particles of the metal salt are distributed through the polymer to form a polymer-particle composite material. For particles including a silver salt, such as a silver halide, the composite material has antibacterial, antifungal, antiviral properties due to the silver ions. A coating of the polymer-particle composite can be applied to a substrate to give the substrate biocidal properties. The charged atomic species of the ionic polymer may be a nitrogen atom, such as a quaternary or ternary nitrogen atom. The ionic polymer may be a cationic polymer such as a partially N-substituted poly(4-vinyl pyridine).

A polymeric coating (including a polymer or composite thereof, such as a polymer-particle composite coating) may be deposited on and/or impregnated into a substrate surface by dip coating, soaking, spray coating, spin coating (for planar substrate), painting, or other coating process. The coating thickness can be readily adjusted, for example by sequentially depositing a plurality of layers to form a single coating. Solution concentration and/or viscosity may be adjusted so as to obtain the desired coating thickness.

Biocidal composites, which may be used as biocidal coatings, include composites having a polymer matrix formed by a cationic polymer, and particles dispersed through the polymer matrix, the particles comprising a biocidal agent. Examples include silver salt particles, where the silver ions are the biocidal agent. Example particles may have a mean diameter between 1 nanometer and 1000 nanometers, in particular between 1 nanometer and 100 nanometers, and the particle size is controllable by the formation parameters. The polymer may be at least partially crosslinked, for example through condensation of hydrolyzable silicon-containing groups, such as silane groups. A composite coating may be covalently linked to a substrate through covalent bonds formed by silicon-containing groups or other functional groups. Composite coatings may be formed on substrates such as is textiles (for example, nylon, cotton, or polyester fibers).

A biocidal (such as a antibacterial and/or antifungal) textile comprises textile fibers and a biocidal coating supported by the textile fibers, the biocidal coating comprising a polymer and particles dispersed through the polymer, the particles comprising a biocide, such as an antimicrobial agent (including antibacterial, antiviral, antifungal, and antiprotozoal agents). The polymer may be an ionic polymer, such as a cationic polymer. The textile may comprise textile fibers, such as synthetic fibers, natural fibers (including plant and animal derived fibers), and combinations thereof. Fibers may include acrylic polymers, acrylate polymers, aramid polymers, cellulosic materials, cotton or other plant-derived fibers, nylon, polyolefin, polyester, polyamide, polypropylene, rayon, animal furs including wool, spandex, silk, viscose, or other known fiber materials. These fibers may be used in textile substrates. The biocide may be silver ions, the particles being a silver compound such as a silver salt. A biocidal coating may be an antimicrobial coating, impeding bacterial and/or fungal and/or viral proliferation, compared with an uncoated substrate.

Examples include polymer-particle composites in which the particles include bromide, phosphorus oxyanions. These ions can give flame retardant properties to the composite, and hence to substrates supporting the composite. Hence, silver salt particle containing composites may be used to prepare textiles and other materials having antibacterial, antifungal, antiviral, and flame retardant properties.

A method of incorporating reactive (e.g. surface binding and/or cross-linking) silane groups into a ionic polymer or copolymer comprises reacting a nitrogen containing polymer (which may be a homopolymer, copolymer or oligomer) with a silane having at least one alkoxy and at least one halide functionality so as to link the silane to the polymer via the nitrogen. Alternatively, a nitrogen containing monomer may be reacted with a silane having at least one alkoxy or at least one halide functionality to link the silane to the monomer via the nitrogen, followed by polymerization of the silane-containing monomer. Example polymers incorporating reactive silane groups include partially N-substituted polyallylamines, and partially N-substituted poly(4-vinyl pyridine). The polymer may have associated counterions.

Biocidal coatings include biocidal composites which may be covalently attached to a substrate surface by reactions of the silane groups of the polymer. A polymer, either as a polymer or composite coating, may be cross-linked by the silane groups of the polymer.

Biocidal coatings may be ion-exchanged, for example by exchanging the counterions with biocidal anions. Ion exchange may introduce anions such as triiodide and various oxyanions like phosphate, carbonate, hypochlorite, bicarbonate, and the like into a polymer or composite material, for example exchanged for an original counterion. Biocidal activity of coatings may be regenerated by treating the coating with a solution having the replenishing active counterion, allowing a surface to have biocidal action restored by wiping with a solution of e.g. triiodide or hypochlorite.

### BRIEF DESCRIPTION OF THE FIGURES

FIG. 1 shows a schematic of particles are formation within an ionic polymer matrix using localized precipitation of a counter-ion;

FIG. 2 is a schematic of an example an on-site precipitation method;

FIG. 3A shows preparation of NPVP (N-substituted polyvinylpyridinium) from poly(4-vinylpyridine);

FIG. 3B shows a schematic for formation of an AgBr/NPVP composite having dual action biocidal properties;

FIG. 4 shows a schematic for incorporating methoxysilane groups into poly(4-vinylpyridine) based polymers;

FIG. 5A shows example nitrogen-containing moieties that may be present in polymers and composites thereof;

FIG. 5B shows example silane-containing groups that may be present in polymers and composites thereof;

FIG. 6 illustrates modification of starting polymers with silane-containing materials to obtain cross-linkable polymers;

FIG. 7 shows modification of monomers to form silane group including monomers;

FIGS. 8A-8D show various preparation schemes for polymers according to examples of the present invention;

FIG. 9A is a schematic showing formation of side-chain silane groups on a polymer;

FIG. 9B is a schematic showing surface binding polymers on a surface;

FIG. 10 illustrates sequential layer by layer deposited (multilayer) covalently linked polymer assemblies of NPVP—Si polymers;

FIG. 11 shows an on-site precipitation technique used to incorporate AgBr particles into the methoxysilane polymer coatings;

FIG. 12A-C illustrate incorporation of iodine into polymer materials;

FIG. 13 is a schematic of I⁻/OCl⁻ ion-exchange;

FIGS. 14A-14B show TEM images of microtomed sections of NPVP(poly(4-vinylpyridine)-co-poly(4-vinyl-N-hexylpyridinium bromide)) composites with AgBr;

FIGS. 15A-15D show further TEM images with particle size histograms;

FIGS. 16A-16E further illustrate antibacterial activity of AgBr/NPVP composites;

FIGS. 17A-17D show SEM image of coated glass surfaces after incubation with P. aeruginosa;

FIGS. 18A-18C show antibacterial activity of AgBr/NPVP—Si (polymer 1b) composites;

FIG. 19 further illustrates the antibacterial activity of AgBr/NPVP—Si (polymer 1b) coated glass slide towards airborne E. coli;

FIGS. 20A-20C further illustrates the antibacterial activity of coated surfaces towards surface borne E. coli;

FIGS. 21A-21D show antibacterial activity persisting for various of rigorously washed substrates;

FIG. 21E-21F show antifungal activity towards Yeast FY250 spread on nutrient agar surface;

FIG. 22 shows an X-ray diffraction pattern of a composite film; and

FIGS. 23 and 24 show ¹H NMR spectra of example NPVP—Si polymers.

### DETAILED DESCRIPTION OF THE INVENTION

Polymeric composite materials are described, including composites comprising a polymer matrix with embedded nanometer and/or micrometer sized particles. The polymer matrix may comprise one or more ionic polymers, the term “polymer” also including copolymers. Examples include the synthesis of antibacterial composites.

Example composites were synthesized using a novel localized on-site (in-situ) precipitation. An example composite comprises two components—a polymeric matrix comprising an ionic polymer, and the in-situ generated nanomicro particles. The particles may comprise an ionic salt such as a silver salt, elemental metal, or other compound.

Applications of materials described herein include biocidal surfaces (including antibacterial, antifungal, and antiviral surfaces), for example for use in health care, food preparation and storage, textiles, hospital surfaces and the lice. Biocidal materials, such as antimicrobial coatings on a substrate, significantly inhibit proliferation of microorganisms, compared with the uncoated substrate. In some examples, biocidal materials have a disinfectant action on the surface of the material and the vicinity due to release of antimicrobial agents. A biocidal material may, for example, be growth-inhibiting and/or disinfecting, in relation to one or more of bacteria, virus, protazoa, or fungus. Example applications include textiles having antibacterial and antifungal properties, food preparation surfaces, medical instruments, medical implants, hospital surfaces, and the like.

Example polymers include novel ternary and quaternary nitrogen containing polymers, having reactive silane groups such as allcylalkoxysilane or alkylchlorosilane groups linked to a ternary or quaternary nitrogen through a linking group. The linking group may be an alkyl or other hydrocarbon group. Alkylsilanol groups attached to the polymer can form covalent Si—O—Si bonds with various surfaces, including glass, fabrics, metals and the like. These groups can also react between themselves, forming a cross-linked polymer coating on the surface. Polymers containing a plurality of silane groups can be prepared from various starting polymers, or from appropriate starting monomers.

Simple techniques were developed for preparing polymers incorporating ternary and quaternary nitrogens, optionally with silane-containing groups or other groups attached thereto, such as alkylalkoxysilane or alkylchlorosilane groups. A silane group is capable of forming covalent and non-covalent links to surfaces such as glass, silicon, ceramics, metals, plastics, nylon, polyester, wood, and paper. A silane groups is also capable of reacting with a similar groups on a nearby polymer, cross-linking polymers having this group.

Surfaces may be coated with silanol group including polymers, such as glass, silicon, ceramics, metals, plastics, nylon, polyester, wood, paper, and the like. Polymers may form covalent and/or non-covalent bonds to the surface. In addition, polymer chains may form covalent and/or non-covalent bonds to neighboring polymer chains resulting in a cross-linked polymer films on the surface being coated. Cross-linked polymer films may be strongly attached to any surface being coated by covalent and/or non-covalent bonds.

Example polymers can be blended with other polymers (such as any polyvinyl polymer, polyesters, polyurethanes and the like) thereby imparting them with surface binding or otherwise adhesive properties. Polymer coatings can be further derivatized to yield new surface chemistries.

Polymers can serve as effective ion-exchange resins, and counterions of the polymer coatings may be ion-exchanged with different functional ions. A polymer may be bound to surface, and ions associate with the polymer can be exchanged with other ions, thereby yielding surfaces with new properties and chemistries.

Polymer coatings may be used to kill both gram positive and gram negative bacteria, for example as a composite with silver-containing or other biocidal particles. Polymer coatings render surfaces antimicrobial for extended periods of time, for example due to membrane disrupting properties, and can yield persistently replenishable and durable antimicrobial surfaces.

Example materials form durable long lasting coatings on various surfaces, such as glass, ceramic, metal, polymer, textiles, paper and wood surfaces. Further, surface-binding polymers allow tailoring of surface energies and surface properties, for example a polymer coating can be used to make a surface hydrophilic or hydrophobic, for surfaces such as glass, ceramic, metal, polymer, textiles, wood etc.

Ions associated with polymers containing ternary and quaternary nitrogen (optionally including alkylalkoxysilane/alkylchlorosilane groups on the nitrogen) can be reacted to yield polymer/inorganic particle composites.

An example composite comprises a polymer and particles, the particles being formed within the polymer by a reaction within the polymer. The polymer can be an ionic polymer, and the particle-forming reaction may include the counter ions. The particles may be nanoparticles or microparticles, and may comprise a metal (such as silver, or a heavy metal), metal salt (such as a silver salt), or other material. The composite may have antimicrobial properties, due to the properties of the polymer and/or the particles.

Example composite materials comprising a cationic amphiphilic polymeric matrix and embedded AgBr nanoparticles were prepared by a relatively simple and novel method, comprising on-site precipitation followed by coordination stabilization of the formed nanoparticles. TEM images clearly showed the presence of highly monodisperse nm sized particles, XRD pattern confirmed the particles were those of AgBr. The composite was shown to have antimicrobial properties. The antibacterial efficacy of the sample increased with the increase in silver concentration in the composite. These anti-bacterial coatings have wide ranging applications in the health industry, food industry, and the like. The on-site precipitation method described may also be used for the synthesis of other types of polymer-nano/microparticle composites such as optical materials, electronic materials, and catalysts.

A process for forming a composite comprises providing a polymer matrix, the polymer matrix including ions, such as counterions; adding a reagent to the polymer matrix, so as to form particles within the polymer matrix due to a chemical reaction between the ions and the reagent, the composite comprising the polymer matrix and the particles. An antibacterial composite comprises particles having biocidal properties, such as silver-comprising particles, within a polymer matrix.

Deposition techniques useful for forming coatings, on textiles and other substrates, include spin coating, dip coating, drop casting, spray coating, flow coating, screen printing, sol-gel processes, and the like.

### Example Polymers and Composite Materials

In some examples, a composite includes at least a first component and a second component. The first component, or matrix, may comprise a polymer, such as an ionic polymer. A polymeric matrix may comprise a homopolymer, a copolymer, oligomer, and may be a blend of two or more polymers. The matrix can include additional components like plasticizers or inorganic fillers to tailor the matrix properties as desired. In some examples, the matrix comprises charged moieties such as anionic or cationic groups associated with at least one of the polymer components of the matrix. The charged moieties have associated counter ions. The charged component of the matrix can be an ionic polymer containing varying amounts of either positive cationic groups and/or negative anionic groups.

The polymeric matrix may comprise a membrane-disrupting contact killing amphiphilic ionic (e.g. cationic) polymer. For example, the polymeric matrix can comprise a cationic polymer, such as a poly(4-vinylpyridinium) based cationic polymer, or other cationic polymer.

Example cationic groups which may be present in ionic polymers include quaternary ammonium groups, biguanide groups, quaternary pyridinium groups, sulfonium groups, phosphonium groups, and imidazolium groups. Example anionic groups include sulfonates, carboxylates, carbonates, sulfates or phosphates.

A negative counter ion associated with the cationic groups of the polymer can be an ion selected from halides, phosphates, sulfates, carbonates, sulfides, acetates, nitrates, nitrites, oxalates, and the like. A positive counter ion associated with the anionic groups of the polymer may be any of the various alkali, alkali earth, transition, and lanthanide or actinide metal ions, other metal ions, or other positive ions. The negative or positive counter ion can also be a charged species comprising two or more elements.

The second component comprises particles embedded inside the polymeric matrix. The particles can vary in size from 1 nm to 10 microns, for example. The particles can comprise, for example, metal compounds such as ionic salts, or metals such as elemental metals. In examples of the present invention, the particles originate due to a chemical reaction between the counter ions associated with the polymeric matrix and the added reagent. Particles can also be modified chemically after formation e.g. by reduction, ligand exchanges substitution, and the like.

The polymeric matrix may optionally have coordinating groups capable of capping and stabilizing the precipitated nanoparticle. It is also possible to further chemically modify the precipitated nanoparticle, for example reduction to elemental metal using a reducing agent.

Composites are described which have two antibacterial modes of action, including a membrane disrupting amphiphilic ionic polymer (such as a cationic polymer), and the release of biocide from particles within the composite. The particles may be Age ion releasing nanoparticles. Example composites were shown to be highly effective in killing harmful microorganisms.

Methods to fabricate highly potent antibacterial composites comprising a cationic polymer matrix having embedded particles (such as silver bromide nanoparticles) were developed, and described further below.

The in-situ particle formation method can be used to make composites of particles with homopolymers, copolymers, and oligomers. It may also be adapted to create mixtures of non-polymeric organic molecules and inorganic nano- or microparticles. No toxic heavy metal catalysts are required, and conditions need not be rigorously controlled to obtain useful results.

Materials and Preparation

A novel technique comprising on-site precipitation was used to make example composites.

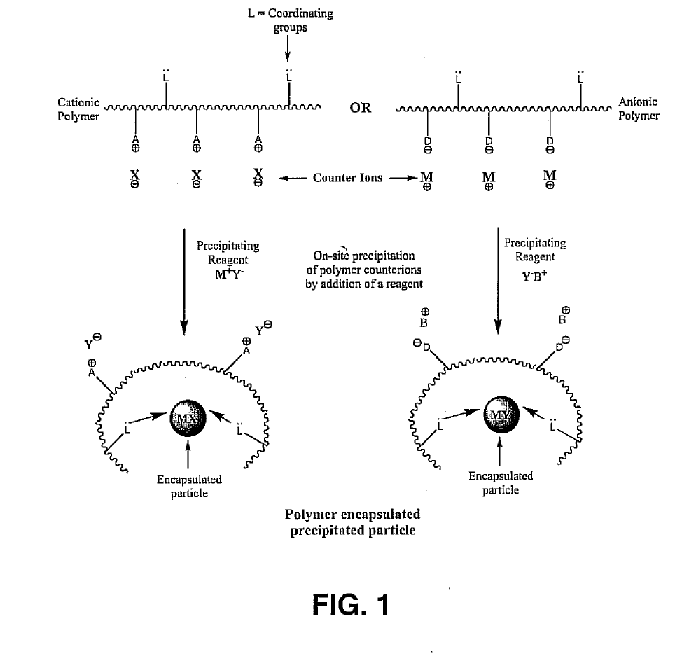

FIG. 1 shows a schematic of an example method, in which particles are formed within an ionic polymer matrix using localized precipitation of a counter-ion.

In this example, the ionic polymer has counter ions associated with the charged groups. The counter ions are precipitated locally on-site by the addition of a suitable precipitating agent such as an ionic salt. Both the ionic polymer and the precipitating agent can be in solution. In this example, the particles of the localized precipitate are stabilized by the steric effect of the polymer chains, and/or the coordinating effect of the coordinating groups of the polymer.

Solubility rules regarding ionic compounds can be used to predict and precipitate desired ionic salt particles. For examples, particles of a sparingly soluble silver salt may be precipitated, such as silver bromide. The resultant precipitate of the ionic compound is stabilized and encapsulated by the surrounding polymer, resulting in a polymer encapsulated particle composite.

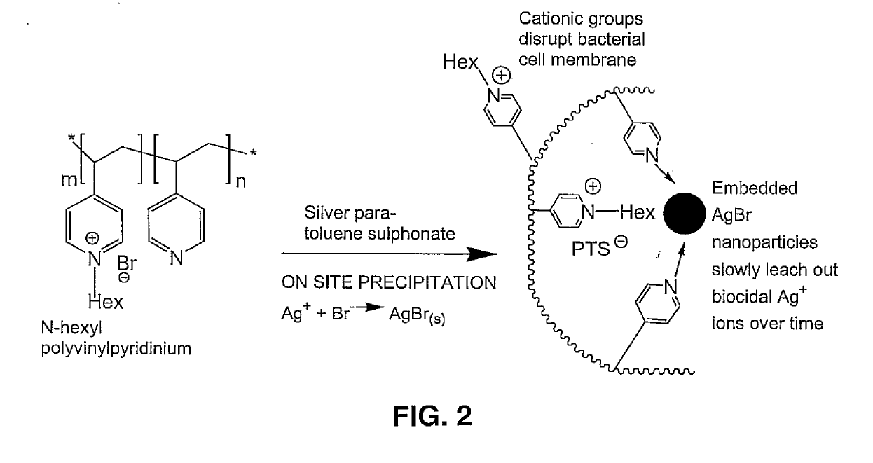

FIG. 2 is a schematic of an example an on-site precipitation method. Bromide anions associated with the polymer side chains of the amphiphilic pyridinium polymer NPVP were precipitated by the addition of a silver salt. The resulting silver bromide nanoparticles are stabilized by the capping and steric effect of the polymer. To the best of our knowledge this is the first example of use of precipitation technique to directly synthesize polymer/nanoparticle composites in a single step. The starting polymer, poly(4-vinylpyridine)-co-poly(4-vinyl-N-hexylpyridinium bromide), NPVP, was prepared by partially N-alkylating the pyridine nitrogens of commercially available polymer poly(4-vinylpyridine) (MW: 60 000).

Two different starting NPVP polymers with 21 and 43% N-allcylation respectively were synthesized. The bromide counter ion (a counteranion) of NPVP was precipitated on site as silver bromide by the slow addition of silver paratoluenesulfonate solution to the polymer solution, yielding composites abbreviated as Agbr/21% NPVP and AgBr/43% NPVP, respectively. For each NPVP polymer, two different AgBr/NPVP composites with silver ion to polymer bromide ion molar ratios of 1:2 and 1:1 were prepared by adding different amounts of silver para-toluenesulfonate. The 1:2 composites yielded clear yellow solutions, whereas the 1:1 composites gave translucent yellow colloidal solutions. Both solutions were stable at room temperature for up to 5 d. Solid AgBr/NPVP composites were obtained by precipitation upon addition to diethyl ether.

Polymer composites having polymerAgPTS weight ratio of 1:1, 1:2, and 1:6 were prepared. The synthesized N-hexyl polyvinylpyridinium polymer (0.5 g) was dissolved in 5 ml of dry nitromethane. AgPTS (0.5 g, 0,25 g, or 0.08 g) was dissolved in 5 ml of dry dimethylsulfoxide. Both the polymer and the AGPTS solutions were cooled to 0° C. on an ice bath. AgPTS solution was then added dropwise to the stirring polymer solution over a time period of 15 minutes. The mixture was stirred for another 30 min at room temperature. The polymeric composite was precipitated out from ethyl ether and was dried under vacuum for 24 hours to yield a yellow colored solid. This solid was then re-suspended in nitromethane and the resultant colloidal solutions were used to cast composite films for antibacterial testing and X-ray diffraction studies.

Addition of AGPTS to the polymer solution yielded a yellow colored colloidal solution of the polymer-nanoparticles composite. As the silver salt is added to the polymer solution having the bromide ion at the polymer side chains, on-site precipitation of AgBr occurs. As AgBr molecules aggregate to form nanoparticles, they are stabilized by the coordination of the pyridine nitrogens and are prevented from aggregating to form larger particles. Steric stabilization of the particles by the alkyl chains of the polymer also contributes in preventing particle aggregation and limits the size of the particles in the nm range.

Four different AgBr containing NPVP composites, 1:1 AgBr/21% NPVP, 1:2 AgBr/21% NPVP, 1:1 AgBr/43% NPVP, and 1:2 AgBr/43% NPVP, were prepared using this approach. Solid AgBr/NPVP composites could be redissolved in methanol, ethanol, nitromethane, or DMSO to give back the colloidal solutions. The composite solutions in methanol were used to form coatings on glass. Solutions of such composite materials may be used to coat various substrates, such as glass, metal, wood, cotton, paper, fibers, textiles, polyester, nylon, spandex, other fabrics, and the like. Substrates may be in the form of fibers (including textiles), planar surfaces, textured surfaces (e.g. to promote coating adhesion), porous surfaces, and the like.

Negligible peeling of deposited AgBr/NPVP composite films from glass substrates was observed, even after adhesion and removal of Scotch tape (3M, Minnesota, Minn.). Glass surfaces are negatively charged due to the presence of surface Si—O— groups. Hence, strong adhesion is expected due to electrostatic attraction between the glass and the cationic polymer. Similarly, good adhesion can be obtained to other substrates, in particular with surface oxygen. Polymers including silane groups may form multiple covalent bond attachments per polymer chain (including —Si—O—Si—). The polymer composites showed strong antibacterial properties, as discussed further below.

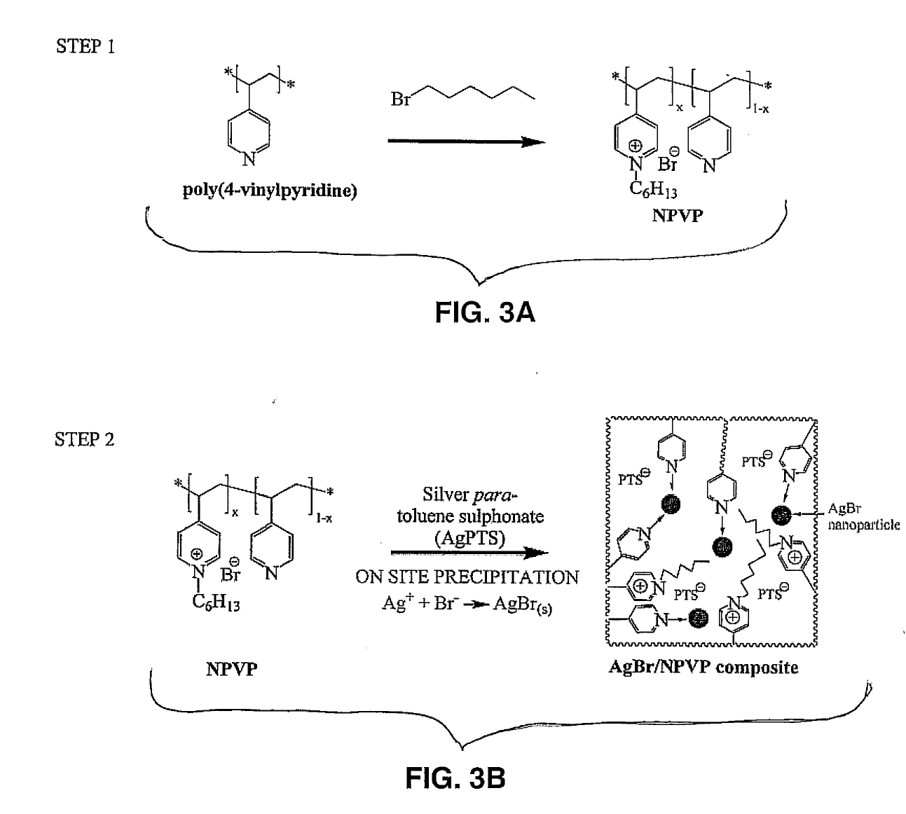

FIG. 3A shows preparation of NPVP (N-substituted polyvinylpyridinium) from poly(4-vinylpyridine). To a 100 ml round bottom flask equipped with a magnetic stirrer were added polyvinylpyridine (1.5 g, 0.014 moles) solution in 25 ml nitromethane and 0.5 equivalents of 1-bromohexane (1.17 g, 0.007 moles). The contents of the flask were stirred at 60° C. for 24 hours. The polymer was isolated by precipitation in ethyl ether and dried under vacuum for 24 hours. The product was characterized by 1H NMR.

Partially N-hexylated polyvinyl pyridine was obtained in nearly 100% yield. The degree of N-alkylation was determined to be 40% based on ¹H NMR peak integrations.

FIG. 3B is similar to FIG. 2, and shows another schematic of an AgBr/NPVP composite.

Examples of the present invention further include surface binding polymers that include one or more surface binding groups. For example, surface binding groups such as silane groups allow binding to oxygen-containing surfaces such as glass or polymer surfaces.

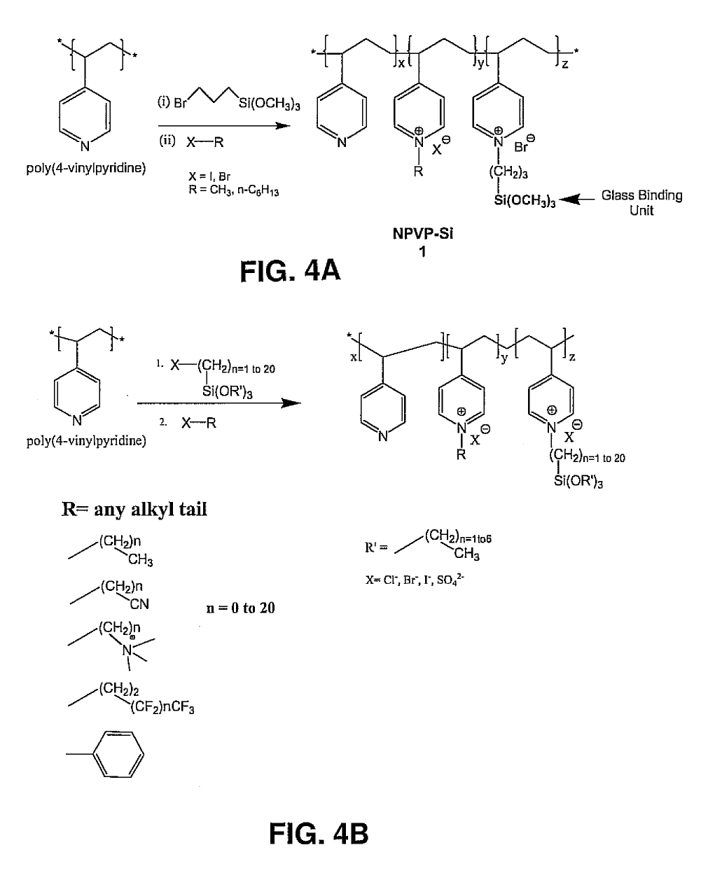

FIG. 4 shows a schematic of a synthetic approach for incorporating methoxysilane groups into poly(4-vinylpyridine) based polymers. In this example, polyvinylpyridine was heated with bromopropyltrimethoxysilane and a haloalkane (such as iodomethane or 1-bromohexane) to yield different polymers. Other silane groups may be incorporated using an analogous approach, such as other alkoxysilane materials. These polymers may be denoted NPVP—Si. By varying the amounts of the haloalkane, various NPVP—Si polymers were synthesized as shown in Table 1 below. All the NPVP—Si polymers were soluble in aprotic polar solvents such as DMSO, nitromethane, and methanol. However after exposure to ambient atmosphere, the polymer chains slowly cross-linked in a couple of days to yield insoluble gels. Hence these polymers were stored under dry nitrogen atmosphere.

TABLE 1

| Polymer | R | x | y | z |
|---|---|---|---|---|
| 1^{#} | — | 85% | — | 15% |
| 1a | C₆H₁₃ | 37% | 50% | 15% |
| 1b | C₆H₁₃ | 4% | 85% | 11% |
| 1c | CH₃ | 37% | 50% | 13% |
| 1d | CH₃ | 4% | 87% | 9% |

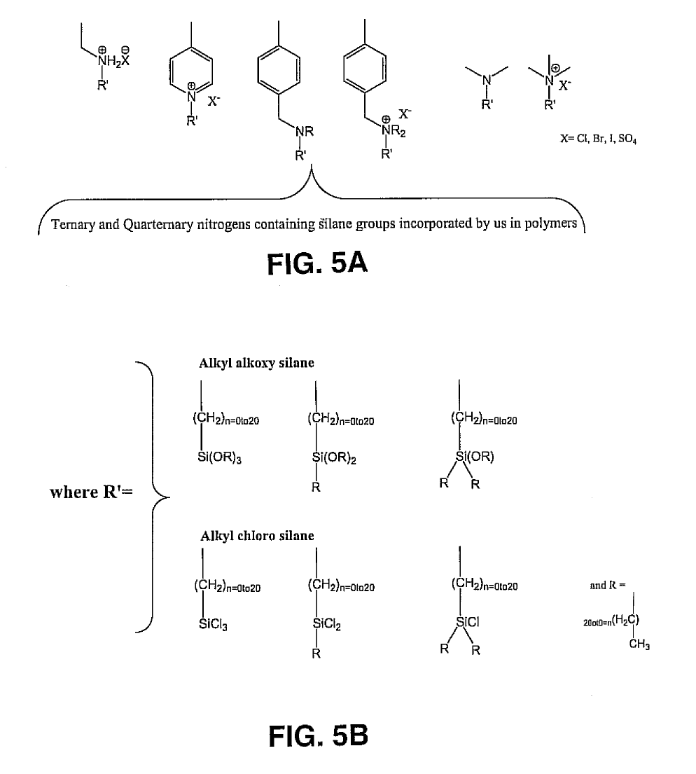

FIG. 5A shows example nitrogen-containing moieties that may be present in polymers and composites thereof. Example polymers according to embodiments of the present invention may include ternary and/or quaternary nitrogens, either as a nitrogen-containing side chain group or nitrogen-containing main chain group. The substituent groups R′ are attached to ternary or quaternary nitrogens. In some examples, R′ may be an alkyl group, or other substituent, including hydrogen. In other examples, R′ may include a hydrolyzable silicon-containing group, such as alkyloxysilane group.

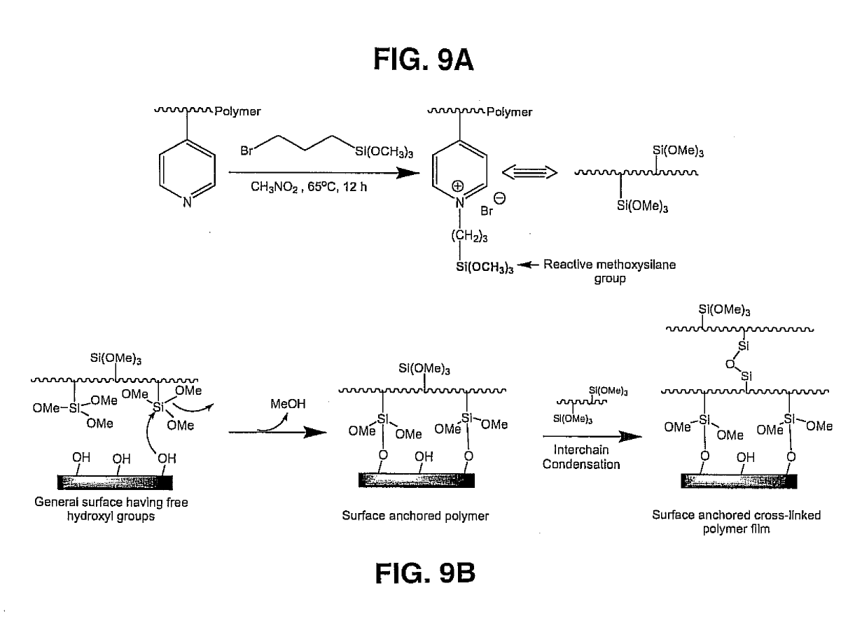

FIG. 5B shows example silane-containing groups that may be present in polymers. The groups R′ may correspond to groups R′ shown in FIG. 9A (though others are possible), and may be included in other example polymers beyond those shown here.

FIG. 6 illustrates modification of starting polymers with silane-containing materials. The starting polymers include ternary or quaternary nitrogens, see also FIG. 9A, where R may be alkyl (such as 1-21 carbon alkyl). The silane-containing material can be X—R′, where R′ may be an alkane-alkyloxy silane, alkane halosilane (e.g. alkane chlorosilane), or other silane-including group, and X may be halide (Cl, Br, I), SO₄, or other functional group. Examples of R′ include those shown in FIG. 9B.

Conducting polymers, such as polypyridine, containing cross-inkable or surface binding groups such as silane groups (e.g. methoxysilane) can be used to form novel materials for electronic and semiconductor application. In addition, salt/metal nano/microparticles may be incorporated into conducting polymers by “on-site precipitation” chemistry described previously to modify their electronic and/or magnetic properties. Applications further include improved electronic conductors, quantum dot formation, metal-conducting polymer nanocomposites, catalysts, light emitting diodes, ion conductors, photovoltaic devices, magnetic media, and the like,

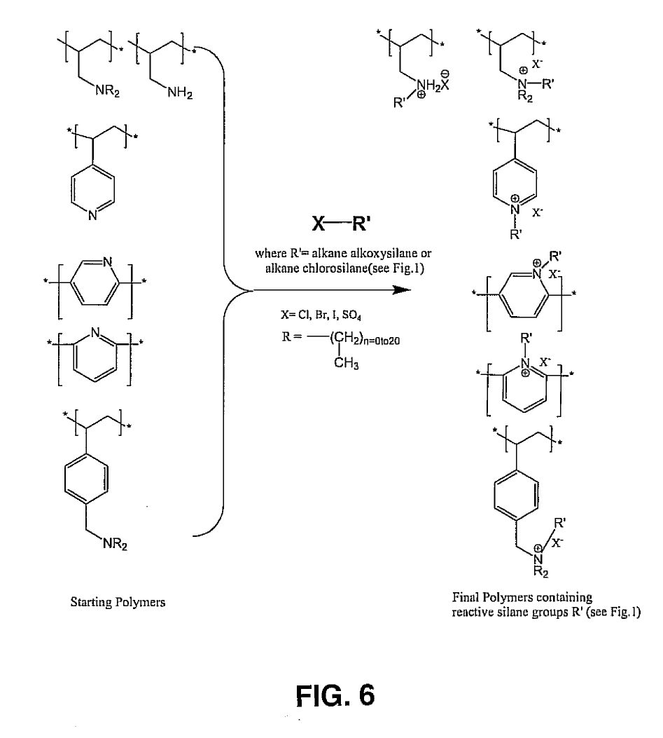

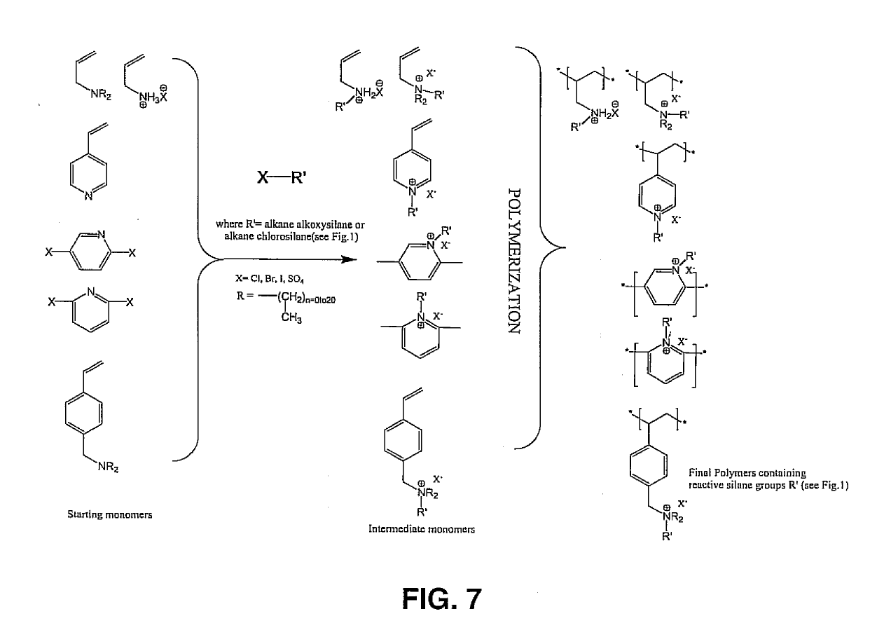

FIG. 7 shows modification of monomers to form silane group including monomers (indicated as “intermediate monomers” in the Figure). These silane-containing monomers may be polymerized (the term polymerization here includes copolymerization with other monomers) to form the example polymers shown.

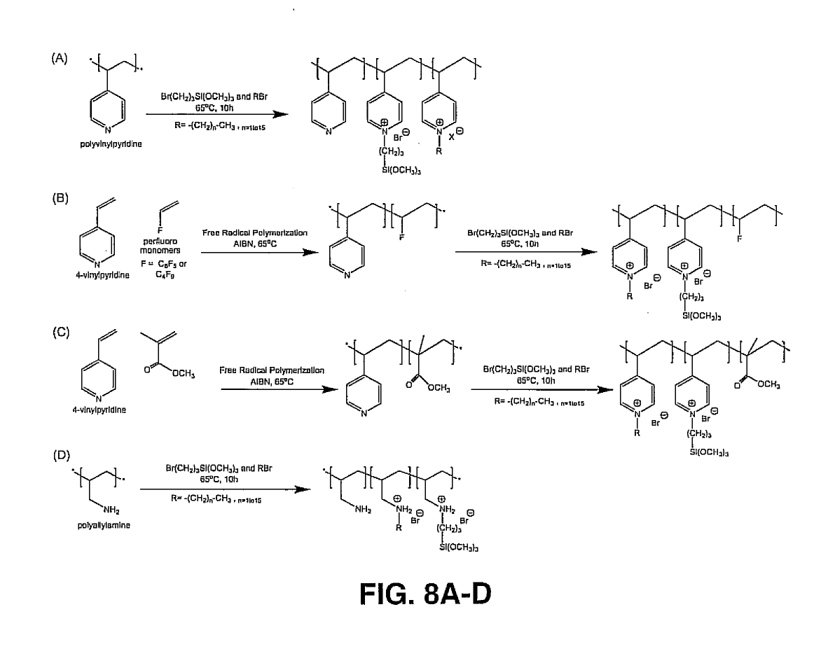

FIGS. 8A-8D show various preparation schemes for polymers according to examples of the present invention.

FIG. 8A is a further schematic for preparation of polyvinylpyridine-based polymers. In this example, commercially available polyvinylpyridine was quaternized with 1-bromopropyltrimethoxysilane and 1-haloalkanes differing in tail lengths. This yielded a library of cationic polymers having surface binding methoxysilane functionalities and antibacterial N-alkylpyridinium groups. Other silane groups may be incorporated using an analogous approach, such as other alkoxysilane materials. The proportion of each functionality was controlled by adding calculated amounts of the silane (˜5-15%) and haloalkane (˜40-70%) reagents, and can be optimized so as to achieve a desired property, such as solubility, surface binding, and/or antibacterial activity.

FIG. 8B is a schematic of polymer preparation by free radical copolymerization of 4-vinylpyridine with two different perfluorinated monomers, perfluorohexene and pentafluorostyrene. The monomer feed ratios were tailored to achieve the optimum copolymer composition for surface binding, antibacterial activity and water repellency. The precursor copolymers were then N-alkylated with 1-bromopropyltrimethoxy silane and/or 1-haloalkanes to introduce surface binding and antibacterial functionalities. Fibrous perfluorinated materials generally show a super-hydrophobic effect, and resist cell/protein adsorption. Hence textiles coated with these perfluorinated polymers can have self-cleaning as well as persistent antimicrobial properties. Other copolymers of vinylpyridine and pentafluorostyrene containing methoxysilane functionalities were prepared as coatings materials for controlling interfacial energies on oxide, metal and semiconductor surfaces, and exhibited high surface energies and formed excellent solvent resistant coatings. Other silane groups may be incorporated using an analogous approach, such as other alkoxysilane groups.

FIG. 8C shows preparation of polymers by free radical copolymerization of 4-vinylpyridine with methyl methylacrylate. The monomer feed ratios can be tailored to achieve the optimum copolymer composition for surface binding, antibacterial activity and polymer toxicity. A series of vinylpyridine-methylmethacrylate copolymers were prepared, and these polymers were discovered to have remarkably low red blood cell toxicity, while retaining high antibacterial potency. These polymers may further be optimized to increase selectivity ratio (antibacterial activity/hemolytic activity). Other example polymers include other copolymers of vinylpyridine with polar monomers such as acrylonitrile, vinyl chloride, styrene, acrylic acid, and the like.

FIG. 8D shows a scheme for preparing polymers from the biocompatible polymer polyallylamine. Polyallylamine is commercially available polymer having side-chain amine functionalities amenable to facile quaternization reactions with various haloalkanes. Polyallyamine derivatives containing surface binding methoxysilane groups and antibacterial alkyl groups were synthesized as shown. Amphiphilic N-alkylated polyallylamine derivatives have been shown to have potent antibacterial activity. Polyallylamine has negligible human toxicity, so is well suited for antimicrobial coatings on textiles. The use of polyallylamine derivatives has been approved by the FDA for treating hyperphosphatemia in patients with chronic renal failure (trade name Renagel® by Genzyme). Moreover polyallylamine derivatives are highly hydrophilic, and hence coatings on textiles should have desirable attributes like comfort, breathability and softness.

Example polymers may be at least partially cross-linked by including functional groups within the polymer. Various cross-linking chemistries may be used. Cross-linking may be induced or speeded up using elevated temperatures, irradiation (e.g. visible or WV irradiation), or other method. In all polymers discussed in relation to FIGS. 8A-D, other silane groups may be used, such as other alkoxysilane groups or halosilane groups.

FIG. 9A is a schematic showing formation of side-chain silane groups on a polymer. The polymer backbone may be any desired type.

FIG. 9B is a schematic of surface binding polymers on a surface. The NPVP—Si polymers can condense with free hydroxyl (-OH) groups on oxide surfaces (such as glass, ceramics, metals, cellulose or cellulosic material, and the like) to covalently anchor the polymer chains to the surface through Si—O—Si linkages. Alkoxysilane groups condense irreversibly with free —OH or other —Si(OR)₃ groups to form strong Si—O—Si linkages. This reaction is facile and is catalyzed by traces of water or added bases or acids. Methoxysilane groups on neighboring polymer chains can further react with each other to form a surface-anchored cross-linked polymer film, which is covalently anchored to the surface.

Coating Formation

For coating formation, solutions of the respective polymers in methanol/water (99/1) were either spin coated or cast by spreading the polymer solution on a clean surface and allowing the solvent to evaporate. The substrates were then placed in oven set at 70° C. for 1-3 hours. The baking step promoted the condensation of the —Si(OMe)₃ groups to yield Si—O—Si covalent linkages both between the polymer and the surface, and in-between the polymer chains. The surfaces were then washed exhaustively with methanol and water for up to 3 days. Finally the silicon or glass pieces dried in nitrogen stream and kept in Teflon boxes for further testing and characterization. In other examples, textiles were coated, as further described below.

Polymers according to examples of the present invention, such as the NPVP—Si polymers discussed above, allow multiple points of covalent linkages to the surface, so that the polymer chains remain anchored to the surface even if one or more of the anchoring linkages break apart. The inter-chain cross linking produces a dense uniform multilayer film structure, compared with a single layer of polymer attached to the surface as obtained with many conventional approaches. Multilayer cross-linked coatings which are covalently anchored to oxide surfaces are expected to have long lasting durability. The coating method described here is fast and can be applied to coat nearly any oxide surface irrespective of shape and size. Covalent attachment of the polymer to the surface does not require any toxic heavy metal catalysts (useful for coating biomedical surfaces), and does not require rigorously controlled conditions like absence of oxygen and water.

The stability of the alkylpyridinium (N⁺—CH₂) bonds was confirmed by stirring a test pyridinium polymer viz. 45%C₆ NPVP at pH 14 and 70° C. for 24 hours. NMR of the sample taken before and after this corrosive treatment showed no significant changes in polymer structure, thereby indicating stability of pyridinium linkages under harsh conditions. Therefore NPVP—Si and similar polymers will likely remain linked to surface even under harsh conditions.

The surface free energy can be adjusted to a desired value by changing the chemical structure of the polymer. For example, polymer 1d was the most hydrophilic due to the presence of high amounts of N-methylpyridinium groups, and had the lowest water contact angle. This approach may be used to permanently modify and control the interfacial properties of an oxide substrate.

Table 2 shows ellipsometry and contact angle measurements of oxide substrates coated with different NPVP—Si polymer. Glass slides and silicon pieces were cleaned and were coated with different NPVP—Si polymer solutions (0.5 wt % in 99/1 methanollwater).

TABLE 2

| | Ellipsometry Thickness | Contact Angle |
|---|---|---|
| Polymer | (nm) | (° degree) |
| No coat | 0 | 11 |
| 1^{#} | 35 | 51 |
| 1a | 29 | 59 |
| 1b | 27 | 63 |
| 1c | 21 | 41 |
| 1d | 31 | 33 |

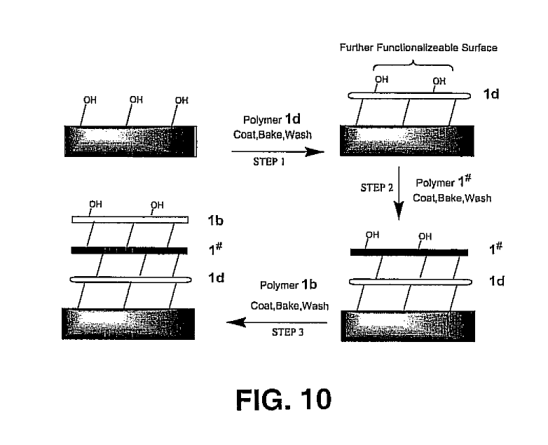

FIG. 10 illustrates sequential layer-by-layer covalently linked polymer assemblies of NPVP—Si polymers 1^{#}, 1b and 1d. Glass surfaces were coated sequentially with polymers 1d, 1^{#} and 1b. Hence, surface polymer films may be built up sequentially, and may comprise a plurality of polymer species. Ellipsometry indicated that the thickness of the coat increased incrementally after each coat/wash step, indicating that the coated polymer was covalently attached to the underlying polymer layer, and was not removed during the washing step. The water contact angle after each coat/wash step was similar to that of the last polymer coated, rather than the underlying polymer. Hence, the surface properties were successively modified by covalently linking a new polymer layer at each step, as summarized in Table 3 below. This approach may be also used to form multilayer polymer films for various applications. Biocidal coatings on substrates may have thicknesses in the range 1 nm-1 micron, such as 10 nm-500 nm. However, thicker coatings may also be prepared if required, and bulk materials may be formed by other processes.

By adjusting the polymer solution concentration, different polymer layer thicknesses may be obtained. Nanometer scale layer-by-layer assemblies of chemically distinct polymers may be formed. These polymer assemblies would have the added advantage of being covalently linked, while having the general applicability of using a wide variety of random copolymers.

TABLE 3

| | Polymer | Ellipsometry | Water Contact Angle |
|---|---|---|---|
| | Coated | Thickness (nm) | (° Degree) |
| STEP 1 | 1d | 23 | 33 |
| STEP 2 | 1^{#} | 37 | 51 |
| STEP 3 | 1b | 59 | 59 |

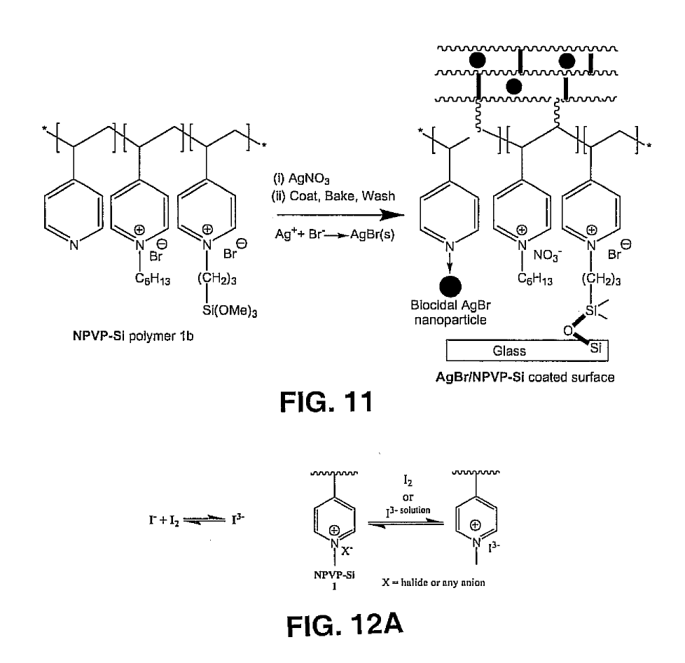

FIG. 11 shows an on-site precipitation technique used to incorporate AgBr particles into the methoxysilane polymer coatings. In this example, NPVP—Si polymer 1b was dissolved in methanol, and an amount of AgO₃ (½ molar w.r.t. polymer bromide ions) was dissolved in water. AgNO₃ solution was then added dropwise to the stirring polymer solution over a time period of 15 min. The bromide ion of the cationic polymer is precipitated as silver bromide upon addition of the silver salt. The solvent composition of final polymer solutions was 99% methanol-l% water. This colloidal solution was coated on glass slides and was baked at 70° C. to give composite films containing AgBr. The coatings had a yellow tinge. Characteristic Ag⁺ 3d binding energy lines were observed at 372.17 and 366.21 eV in a high resolution XPS scan of AgBr/NPVP—Si 1b coated surfaces. The line positions were consistent with those reported in literature for AgBr.

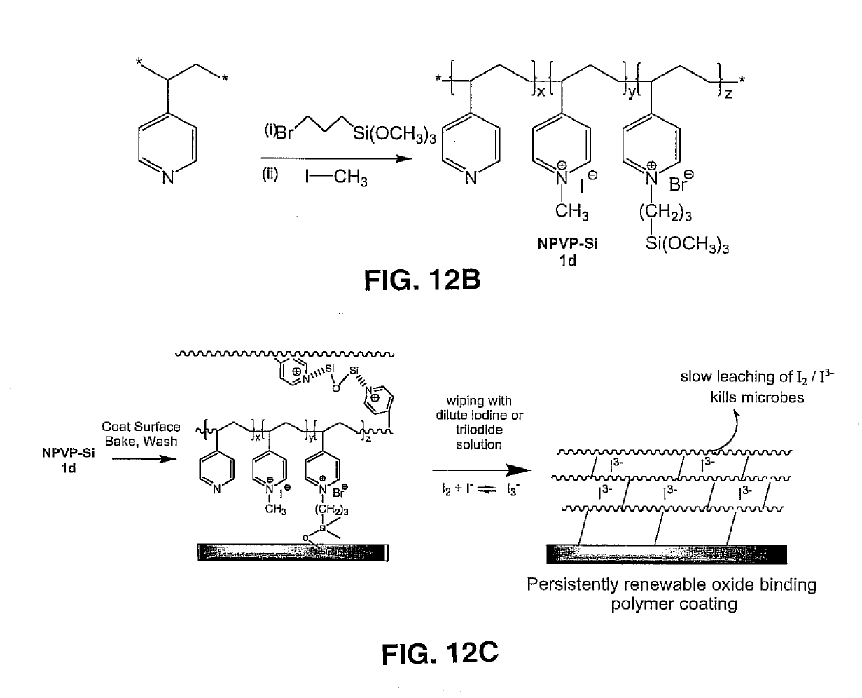

FIGS. 12A-12C illustrate incorporation of iodine into polymer materials. Iodine has antimicrobial properties. FIG. 12A shows exchange of counter ion X— with the triiodide ion, in this example using NPVP—Si polymer 1. FIG. 12B illustrates formation of the silane-including polymer NPVP—Si polymer 1. FIG. 12C illustrates that wiping a coated surface with dilute iodine or triiodide solution provides a persistently renewable polymer coating.

Antimicrobial activity of coated surfaces can thereby be constantly replenished by just treating/wiping surface with dilute iodine solution or ion-exchanging with triiodide solution. This yields persistently renewable polymer and composite coatings for any surface, such as glass, silicon, ceramic, plastic, metal, textiles, nylon, polymers such as polyester, cellulosic material, wood, paper, and the like. Polymer substrates may include fibrous and planar (e.g. sheet) substrates.

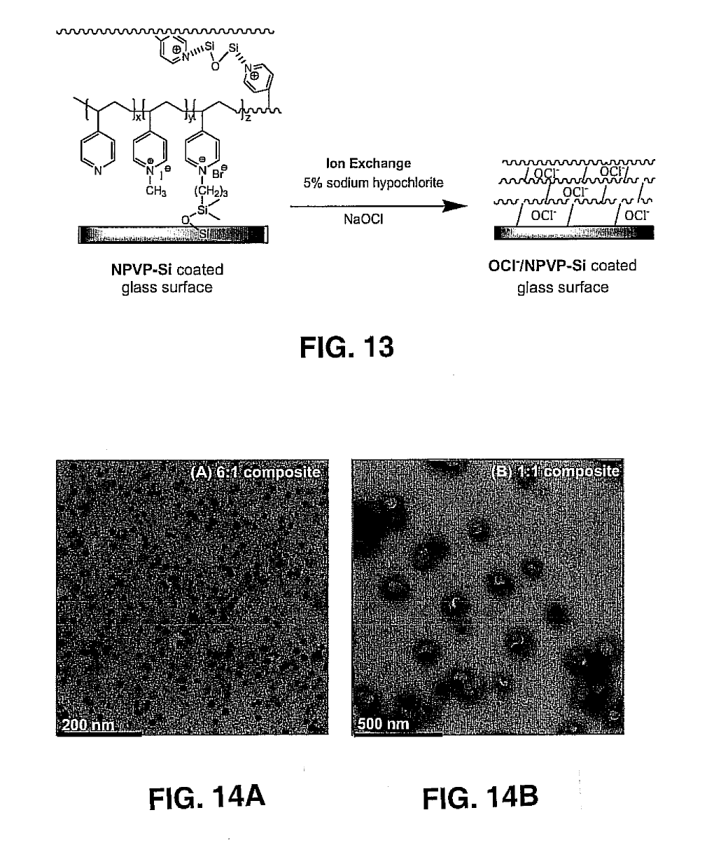

FIG. 13 is a schematic of I⁻/OCl⁻ ion-exchange on NPVP—Si 1d coated glass surfaces. The hypochlorite anion is a well known oxidizing species which is known to kill nearly every type of microorganism. NPVP—Si polymer 1d was coated on glass surfaces as described before, and polymer coated glass/silicon pieces were dipped in 5% sodium hypochlorite for 2h to enable I⁻/OCl⁻ ion exchange. These surfaces were found to be antibacterial, but over time the polymer was degraded by the highly oxidizing OCl— ions, as shown by FTIR of ion-exchanged silicon surfaces. A less oxidizing anion like triiodide (see FIGS. 12A-12C), which is also highly biocidal, does not induce such degradation. However, this approach is possible using other polymer materials.

Examples of the present invention include combinations of cationic polymers and oxidizing anions, such as iodine, triiodide, or OCl⁻.

Polymer Composite Imaging and Particle Size Distributions

For TEM imaging, a solid polymer composite was embedded in Epon resin and thin slices of the sample were sectioned off using the ultracut microtome. The sections were collected on a carbon coated 300 mesh size copper TEM grid and were observed under the electron microscope.

FIGS. 14A-14B show TEM images of microtomed sections of NPVP (poly(4-vinylpyridine)-co-poly(4-vinyl-N-hexylpyridinium bromide)) composites with AgBr. FIG. 14A shows a NPVP:AgBr composite having 6:1 polymer:AgPTS weight ratio, and FIG. 14B shows a composite having 1:1 polymer:AgPTS weight ratio. The TEM images of the composite microtomed sections clearly indicate the presence of spherical nanoparticles. Highly monodisperse AgBr nanoparticles with an average particle size of 13 nm were obtained for the 1:6 composite. The 1:1 composite gave larger, somewhat non-spherical particles with an average size of 75 nm. A 1:2 composite showed monodisperse nanoparticles with an average particle size of 14 nm.

The increase in particle size with the increase in silver:polymer ratio was attributed to a decrease in the coordinating nitrogen:silver ratio. Possibly, as the proportion of free coordinating nitrogens decreases relative to the amount of Ag, there is lesser stabilization of the growing AgBr particles. This leads to increased aggregation of growing AgBr particles resulting in larger particle size. X-ray microanalysis of the composite sections showed significant amounts of silver and bromine.

Hence, particle size within a composite is controllable, for example to control release rates of biocidal agents from the particles.

The effect of the degree of allcylation on the particle size was investigated using 21% alkylated NPVP instead of 43% NPVP as the base polymer. For 1:1 silver: bromine molar ratio, smaller nanoparticles were observed for 23% NPVP than for the 43% NPVP (FIG. 3 d-e). This is attributed to a higher number of coordinating pyridine groups in 23% NPVP, which would result in higher stabilization of the growing nanoparticles. This would lead to decreased aggregation and hence smaller sized nanoparticles. Hence, we can control the particle size by controlling the degree of alkylation of polyvinylpyridine (Table 1). Interestingly there was little size difference between the 23% and 43% NPVP for 1:6 and 1:2 silver: bromine molar ratios. Hence, the particle size of AgBr was controllable by changing the percentage of alkylation or the ration of silver to polymer.

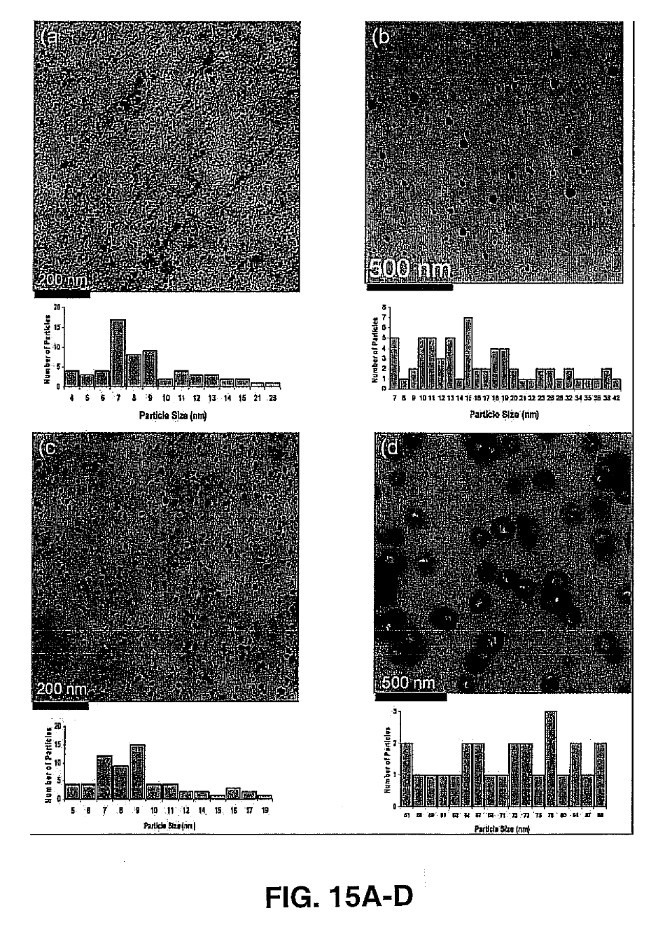

FIGS. 15A-15D show further TEM images with particle size histograms of microsections of the solid AgBr/NPVP composites. FIG. 15B shows 1:2 AgBr/21% NPVP, FIG. 15A shows 1:1 AgBr/21% NPVP, FIG. 15C shows 1:2 AgBr/43% NPVP, and FIG. 15A shows 1:1 AgBr/43% NPVP.

TEM images clearly indicate the presence of spherical nanoparticles embedded inside the solid polymer and suggest that precipitation is taking place on-site very close to the polymer chains FIG. 2). If AgBr had precipitated in solution away from the polymer chains, high and uniform distribution of nanoparticles throughout the polymer matrix would not have been expected. Since the precipitation takes place in the vicinity of the polymer chains, the growing AgBr nanoparticles are stabilized and prevented from aggregating by the capping action of the coordinating pyridine groups. Steric isolation by the comb-shaped polymer also helps in the stabilization of the nanoparticles. Similar stabilization of nanoparticles in polymer matrixes has been documented previously for metal nanoparticles in polysiloxane solutions. Both the degree of polymer N-alkylation (21 versus 43%) and bromide to added silver molar ratio (1:1 versus 1:2) had significant effect on the size of embedded nanoparticles (FIG. 15 A-D, Table 5).

The lower the degree of N-alkylation of the polymer (21 versus 43%), the smaller the resulting nanoparticles. This can be attributed to a higher proportion of coordinating pyridine groups in 21% NPVP over 43% NPVP, which would result in higher capping efficiency for the growing nanoparticles. For the NPVP polymer, the lower the Ag+ to Br— (polymer) ratio (1:2 versus 1:1) the smaller the nanoparticles. This is presumably a result of lower AgBr/capping agent ratio in the former. Interestingly, there appears to be a lower size limit for the AgBr particles since the 1:2 composites of both 21% NPVP and 43% NPVP have similar sized particles.

Table 5 below shows average particle sizes in nanometers for AgBr/21% NPVP and AgBr/43% NPVP composites, the percentage being % N-alkylation, and standard deviations given in parentheses.

TABLE 4

| Ag/Br ratio | 43%: Average AgBr | 21%: Average AgBr |
|---|---|---|
| in composite | size (nm) | size (nm) |
| 1:2 | 10 (4) | 9 (4) |
| 1:1 | 71 (11) | 17 (9) |

Antibacterial Properties of Polymers and Composites

Highly potent antibacterial composites were synthesized, comprising a cationic polymer matrix having embedded silver bromide nanoparticles. AgBr is more soluble than elemental silver (Kₛₚ=5×10⁻¹³), and affords a higher concentration of Ag⁺ ions in the surrounding medium. Poly(4-vinylpyridinium) based cationic polymers are known to have potent antibacterial action towards both gram positive and gram negative bacteria. Hence, example composites may have two antibacterial components, a membrane disrupting contact killing amphiphilic cationic polymer, and Ag⁺ ion releasing nanoparticles. In-situ precipitation of AgBr was used to synthesize the polymer-nanoparticle composite. Other silver compounds, such as other silver halides, may also be used. In one experiment, a modified Kirby Bauer disc diffusion technique was used to probe the bactericidal effect of the composites. Identical sized filter papers were coated with same amounts of AgBr/43% NPVP composites solutions in methanol and were dried. These filter papers were then placed on bacteria-inoculated agar plates and were visualized for antibacterial activity after incubating overnight. The bacteria spread on agar plates closely resemble real world situations in which pathogenic bacteria are often present on receptive nutrient surfaces in biomedical implants, medical devices or food packaging surfaces.

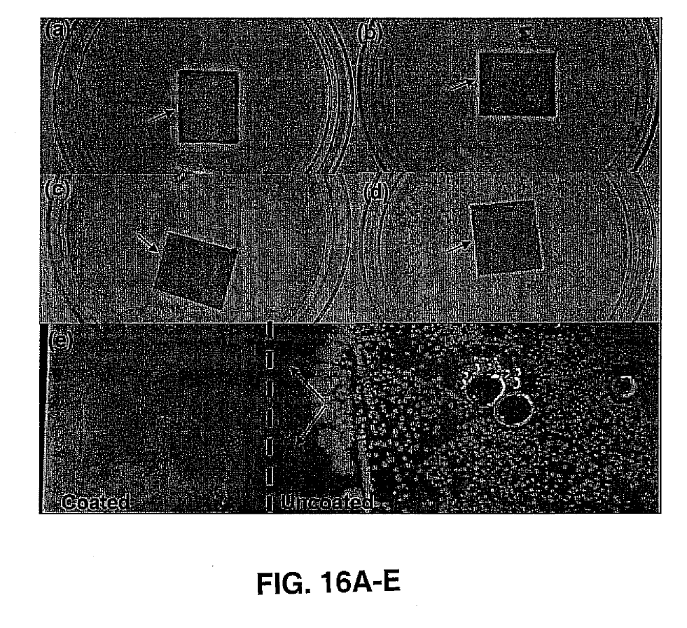

The NPVP/AgBr composites placed on the bacteria inoculated surfaces killed all the bacteria under and around them (FIGS. 16A-16D). Distinct zones of inhibition (clear areas with no bacterial growth) were observed around the composite samples for both E. coli and B. cereus}. High bacterial growth as indicated by bacterial growth lawn (large indistinguishable collection of colonies where colonies have merged together to form one field of bacteria) was observed everywhere else. Also no bacterial growth was observed under/within the composites. Controls consisting of sodium para-toluene sulfonate and 21% and 43% NPVP impregnated filter papers exhibited no zones of inhibition. The poor solubility of 43% NPVP in LB broth, coupled with slow diffusion of the comb-shaped polymer macromolecule through solid agar results in the lack of a zone of inhibition. On the other hand Ag⁺ ions are highly soluble in LB broth and can diffuse readily, thereby exhibiting clear zone of inhibition. However 43% NPVP does kill bacteria in presence of liquid LB broth, although much less effectively than AgBr/NPVP composites (see Tables 6 and 7).

These composites were also tested as antibacterial coatings on surfaces. The antibacterial activity of coated glass slides towards airborne E. coli was tested. Glass slides were partially coated by evaporating 3×50 μL of 5 wt % composite solution in methanol. Airborne E. coli bacteria were then sprayed on the surface of the coated discs and bacterial growth was visualized after overnight incubation in LB agar. No bacterial growth was observed on top of the coatings, as well as adjacent to the coatings (zone of inhibition) as shown in FIG. 16E.

Bacterial growth was seen on uncoated glass surface as indicated by the presence of colonies. In both the Kirby Bauer testing with composite-coated paper and glass slide testing, the observed zone of inhibition is a result of the leaching of active biocidal species Ag⁺ ion from the embedded AgBr nanoparticles present in the composite into the surrounding aqueous medium. The presence of the inhibition zone clearly indicates that the mechanism of the biocidal action of the composite is not merely due to membrane disruption by the ampiphilic NPVP but also due to the leached Ag⁺ ion. The size of the zone of inhibition for different AgBr/43% NPVP composites are given in Table 5. Interestingly, the size increased with decrease in the size of the AgBr nanoparticles. We attribute this to higher rate of leaching from the smaller particles due to their higher surface to volume ratio. Thus, it is possible to control the leaching rate of Ag⁺ ion by varying the size of the embedded AgBr particles.

The thicknesses of the zones of inhibition for different Ag—NPVP composites are given in Table 5 below. Interestingly the thickness of the zones of inhibition increased with decrease in the silver to polymer ratio i.e. from 1:1 to 1:2. This was attributed to smaller sized nanoparticles in the 1:2 composite (13 nm) as compared to the 1:1 composite (72 nm). Smaller sized particles would have larger effective surface area of AgBr and thereby lead to higher concentration of Ag⁺ in the surrounding medium, as the dissolution of AgBr to produce Ag⁺ is a surface process. Hence we have been able to tune the rate of release of Ag⁺ by controlling the particle size.

Table 5 shows the correlation of the thickness of zones of inhibition for two different Ag-43%NPVP coated paper squares placed on bacteria inoculated LB-agar plates with the AgBr particle size in the composite.

TABLE 5

| | AgBr | Zone of | Zone of |
|---|---|---|---|
| | particle | inhibition | inhibition |
| Ag - 43% | size | E. Coli | B. cereus |
| composite | (nm) | (mm) | (mm) |
| 1:2 | 15 | 3 | 2 |
| 1:1 | 72 | 2 | 1 |

The dual action antibacterial properties of the composite were also investigated, i.e. the bactericidal effect of Age and membrane disrupting amphiphilic cationic polymer. A series of known weights of the 1:2 silver composite and the 43% NPVP polymer were each incubated with increasing amounts of E. coli and B. cereus in aqueous LB broth. After 18 hours of incubation, bacterial growth was measured by visually inspecting the turbidity of the solutions and then plating 100 μL of the incubated LB broth on LB-agar growth plates. Bacterial colonies were then counted after incubating the plates overnight. The results are shown in table 6 and 7 below. The 1:2 composite killed/inhibited E. Coli at concentrations of at least 500,000 bacteria per mg composite, whereas the 43% NPVP polymer was effective at lower bacterial concentration of at least 50,000 bacteria per mg polymer. Hence the silver containing NPVP composite had higher antibacterial activity than 43% NPVP polymer for both E. Coli and B. Cereus. This observation supported the fact that both mechanisms of action are operating in the silver-polymer composites.

Table 6 shows comparison of the antibacterial activity of Ag-43%NPVP and 43%NPVP towards gram negative E.coli .

TABLE 6

| | Weight | Total | LB broth | |
|---|---|---|---|---|
| | taken in 2 ml | bacteria | turbidity after | Colonies on |
| | LB broth | added per | 18 hours of | agar plate after |
| Sample | (mg) | mg solid | incubation | plating 100 μL |
| 1:2 Ag- | 6.3 | 50000 | Clear | none |
| NPVP | | | | |
| 1:2 Ag- | 8.3 | 500000 | Clear | none |
| NPVP | | | | |
| 1:2 Ag- | 6.1 | 5000000 | Turbid | lawn |
| NPVP | | | | |
| 43% NPVP | 7.1 | 50000 | Clear | none |
| 43% NPVP | 6.7 | 500000 | Turbid | lawn |
| 43% NPVP | 6.5 | 5000000 | Turbid | lawn |
| PVP control | 9.3 | 50000 | Turbid | lawn |

Table 7 shows a comparison of the antibacterial activity of Ag-43%NPVP and 43%NPVP towards gram positive B. cereus.

TABLE 7

| | Weight | Total | LB broth | |
|---|---|---|---|---|
| | taken in 2 ml | bacteria | turbidity after | Colonies on |
| | LB broth | added per | 18 hours of | agar plate after |
| Sample | (mg) | mg solid | incubation | plating 100 μL |
| 1:2 Ag- | 5.9 | 50000 | Clear | none |
| NPVP | | | | |
| 1:2 Ag- | 6.8 | 500000 | Clear | none |
| NPVP | | | | |
| 1:2 Ag- | 7.2 | 5000000 | Clear | 15 |
| NPVP | | | | |
| 43% NPVP | 6.9 | 50000 | Clear | none |
| 43% NPVP | 7.8 | 500000 | Clear | 33 |
| 43% NPVP | 6.8 | 5000000 | Turbid | lawn |
| PVP control | 8.5 | 50000 | Turbid | lawn |

FIGS. 16A-E further illustrate antibacterial activity of AgBr/NPVP composites. Zone of inhibition is indicated by arrows. FIG. 16A shows 1:2 AgBr/43% NPVP composite-coated paper placed on the LB agar plate inoculated with E. coli showing a comparatively large zone of inhibition, FIG. 16B shows 1:1 AgBr/43% NPVP composite showing a comparatively small zone of inhibition, FIG. 16C shows 1:2 AgBr/43% NPVP composite-coated paper placed on the LB agar plate inoculated with B. cereus showing a comparatively large zone of inhibition, and FIG. 16D shows 1:1 AgBr/43% NPVP composite showing a comparatively small zone of inhibition. FIG. 17E shows a glass slide coated with 1:1 AgBr/21% NPVP and sprayed with airborne E. coli mist also exhibiting a zone of inhibition. E. coli colonies can be seen in uncoated area.

When immersed in an aqueous culture medium, the composites were found to prevent bacterial growth over time periods of 17 days or more. Leaching of silver ions does not occur when the composite is in a generally dry environment, so that antibacterial action can be retained for much longer time periods.

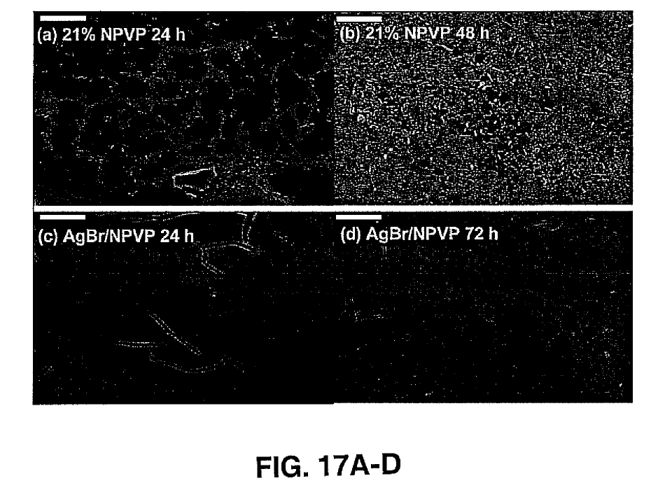

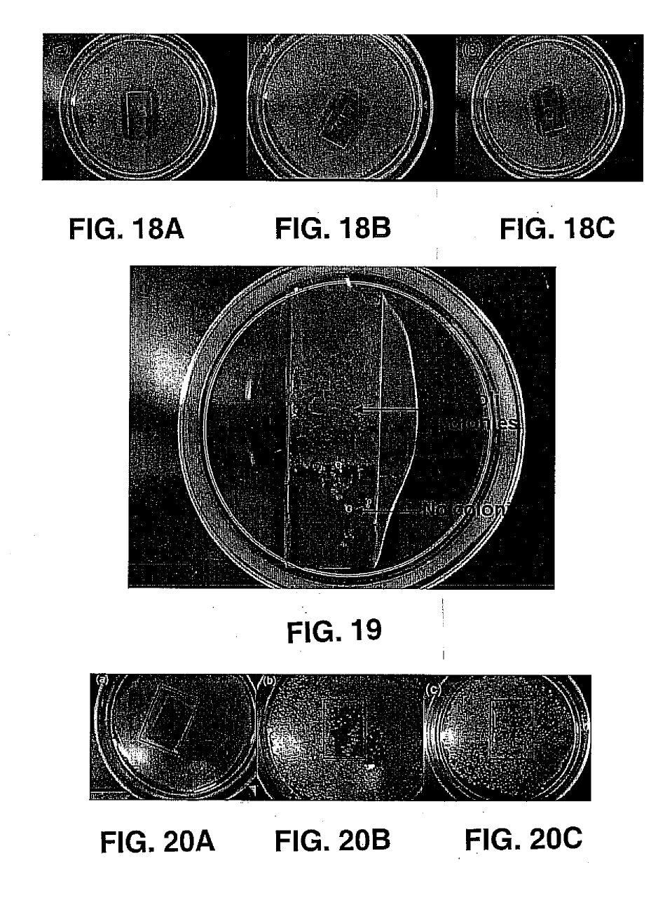

FIGS. 17A-17D show SEM image of coated glass surfaces after incubation with P. aeruginosa. FIGS. 17A and 17B show biofilm on 21% NPVP-coated glass surfaces after 24 and 48 h incubation. Dense collection of rod-shaped bacteria can be seen colonizing the surface. FIGS. 18C and 18D show no biofilm formation observed on 1:1 AgBr/21% NPVP coated glass surfaces even after 72 h incubation. Scale bar is 10 micron.

The cationic polymer 21% NPVP initially kills bacteria in immediate contact with its surface due to its membrane-disrupting effect. However, dead cells and cellular debris adhering to the positively charged polymer surface would attenuate any further membrane-disrupting action. Moreover, dead cells and debris on the surface of the polymer provide an organic conditioning layer, a necessary first step in biofilm formation. Hence, a compact biofilm forms on 21% NPVP-coated surfaces. In the case of AgBr/NPVP-coated surfaces, constant diffusion of the Ag⁺ ion creates an antibacterial zone extending some distance beyond the immediate surface and hence prevents biofilm formation. The composite film prevents biofilm formation, unlike the polymer alone.

Table 8 below further illustrates antibacterial activity of AgBr/polymer nanocomposites towards methicillin resistant S. aureus after exposure to mammalian fluids.

TABLE 8

| Sample | Human Serum | Human Saliva | Human Blood |
|---|---|---|---|
| AgBr/Polymer | Bactericidal¹ | Bactericidal¹ | Bactericidal¹ |
| Nanocomposites | at 150 μg/ml; | at 100 μg/ml; | at 200 μg/ml; |
| | Bacteriostatic² | Bacteriostatic² | Bacteriostatic² |
| | at 100 μg/ml | at 50 μg/ml | at 100 μg/ml |
| Pyridinium Polymer | Inactive | Bactericidal¹ at | Inactive |
| Alone | | 5000 μg/ml | |

¹Bactericidal: <10 colonies observed after plating 100 μL; sample kills >99.9999% bacteria at the given concentration.
²Bacteriostatic: ˜300-400 colonies observed on plating 100 μL; sample kills/inhibits growth of >99% bacteria.

FIG. 18A-18C show antibacterial activity of AgBr/Polymer 1b (NPVP—Si polymer 1b discussed above) coated glass slides towards surface borne E. coli . FIG. 18A shows Day 1. FIG. 18B shows Day 3, and FIG. 18C shows Day 5.

FIG. 19 further illustrates the antibacterial activity of AgBr/NPVP—Si 1b coated glass slide towards airborne E. coli. These polymer-AgBr composites covalently bind to glass surfaces by their reactive methoxysilane groups. Thus coatings of these polymers remained attached to surfaces even after washing with solvents for two days. The antibacterial activity was retained even after these washing steps, indicating that these coatings can be used for creating durable, wash resistant antimicrobial surfaces.

Glass slides were partially coated with AgBr/NPVP—Si 1b solutions, an airborne E. coli bacteria were then sprayed on coated surfaces and bacterial growth was visualized after overnight incubation in LB agar. No colony growth was observed on the part of glass slide which was coated. Bacterial growth was seen on uncoated glass surface as indicated by the presence of colonies. The AgBr/NPVP—Si coatings apparently kill bacteria by biocidal Ag⁺ ion diffusion.

FIGS. 20A-20C further illustrates the antibacterial activity of coated surfaces towards surface borne E. coli . FIG. 20A shows the effect of a AgBr/Polymer 1b coating (within the indicated box), FIG. 20B shows an ion-exchanged Polymer 1d-OCl⁻ coating, and FIG. 20C shows uncoated glass with no antibacterial effect.

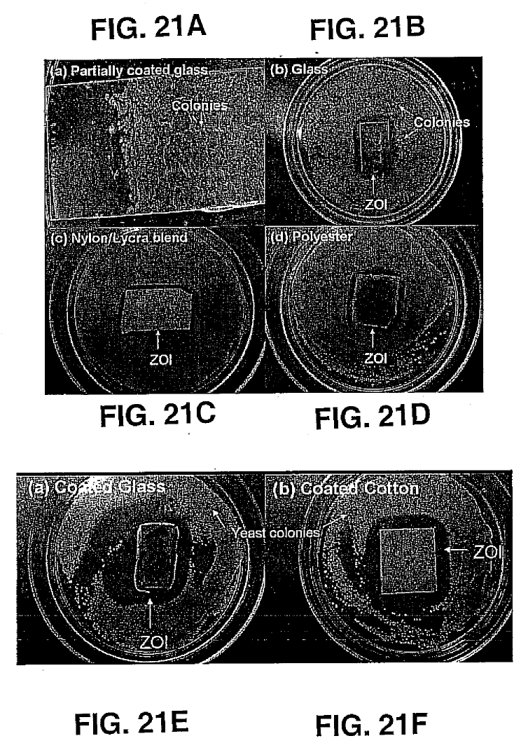

FIGS. 21A-21D show antibacterial activity various of rigorously washed substrates (glass and commercially available textiles) towards gram negative E. coli. ZOI stands for zone of inhibition, the region in which no bacterial growth was observed due to slow diffusion of biocidal silver ions from the composite coatings. The bromide counter anion of polymer la was precipitated on-site as silver bromide by the slow addition of silver nitrate solution to the polymer solution, yielding an AgBr nanoparticle-polymer composite abbreviated as AgBr/Polymer la. A colloidal solution was coated on coated on various surfaces (e.g. spin coating for glass, metals; dip coating for textiles) and balled at 70° C. for 1 hour to give polymer-nanoparticle composite films containing antibacterial AgBr. Surface analysis techniques indicated that the coatings were retained even after rigorous washings with solvents and detergents.

These AgBr composites remained tightly anchored to textile surfaces absent of free hydroxyl groups (including nylon, spandex, and polyester). Inter-chain polymer cross-linking upon baking may lead to locked, interpenetrating networks of polymer-fiber chains, thereby effectively anchoring the polymer coating on these fibers fence, these polymer formulations can be used to give antibacterial coatings on nearly any kind of non-spun monofilament or woven textiles. Antibacterial properties were imparted to commercially available cotton, nylon, spandex, and polyester fabrics. Antibacterial activity was retained after rigorous washing cycles lasting several days with methanol, water and detergents at elevated temperatures.

The textile coating process may use a relatively low temperature, even room temperature, for cross-linking, such as between 20 F and 80 F. The cross-linking temperature may be adjusted according to the temperature sensitivity of the substrate.

FIGS. 21E-21F show antifungal activity towards Yeast FY250 spread on nutrient agar surface. FIG. 21E shows a glass surface coated with AgBr/Polymer la composite showing zone of inhibition (ZOI) with no fungal growth around the coated piece. FIG. 21F shows a cotton textile fabric coated with AgBr/Polymer la composite also showing zone of inhibition. Both samples were rigorously washed with methanol/detergent/water prior to testing, thereby indicating that antifungal coating was durable and long-lasting. FTIR spectroscopy indicated that the polymer coating was chemically intact after the rigorous methanol/detergent/water washing, XPS surface analysis also indicated presence of substantial polymer layer after wash steps.

The antibacterial activity lasted for more than a week (testing was stopped after 7 days), indicating that these coatings can be used to generate long lasting antibacterial surfaces. Moreover, the methodology described above was applicable to surfaces irrespective of chemical identity, and was demonstrated to yield potent long lasting antibacterial coatings on oxide, metal and textile surfaces. In some cases, polymers without surface-binding or cross-linking silane groups were removed by the rigorous washing steps.

Applications of such materials are discussed further below.

Materials and Instrumentation

All reagents were used without further purification. Polyvinylpyridine (Mol. Wt.=160,000), 1-bromohexane (99+%) and silver para-toluenesulphonate (AGPTS) (99+%) were purchased from Aldrich. Nitromethane (ACS grade) was purchased from Acros Organics. Bacterial growth media and agar were purchased from Difco. ¹H NMR (300 MHz) was recorded on a Brucker DPX-300 instrument. A Reichart-Jung Ultracut E Microtome was used for sectioning the composite samples for TEM imaging. TEM imaging and X-ray microanalysis were done on a JEOL JEM 1200EXII electron microscope equipped with an energy dispersive X-ray system operating at an accelerating voltage of 80 kV. X-ray diffraction spectra were recorded on a Philips X'Pert—MPD analytical X-ray equipment.

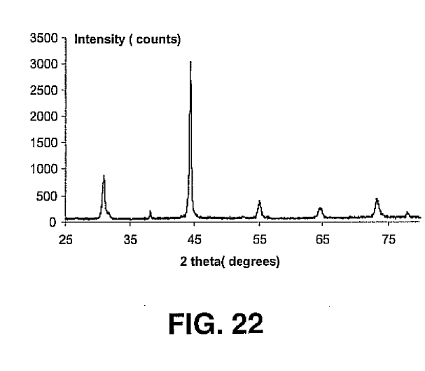

FIG. 22 shows X-ray diffraction patterns of the 1:1 composite film. To establish whether the nanoparticles were those of AgBr or elemental silver, X-ray diffraction spectra of a 1:1 composite film cast on an aluminum sample holder was recorded. The XRD diffraction pattern indicated the presence of AgBr, rather than that of elemental silver. Thus the AgBr nanoparticles formed are stable and are not chemically reduced to elemental silver during the synthetic procedure. This may be desirable, since AgBr nanoparticles yield a much higher concentration of Ag⁺ ions in the surrounding medium than elemental Ag nanoparticles since AgBr (Kₛₚ=5×10⁻¹³) is more soluble than Ag, giving a more effective antimicrobial material.

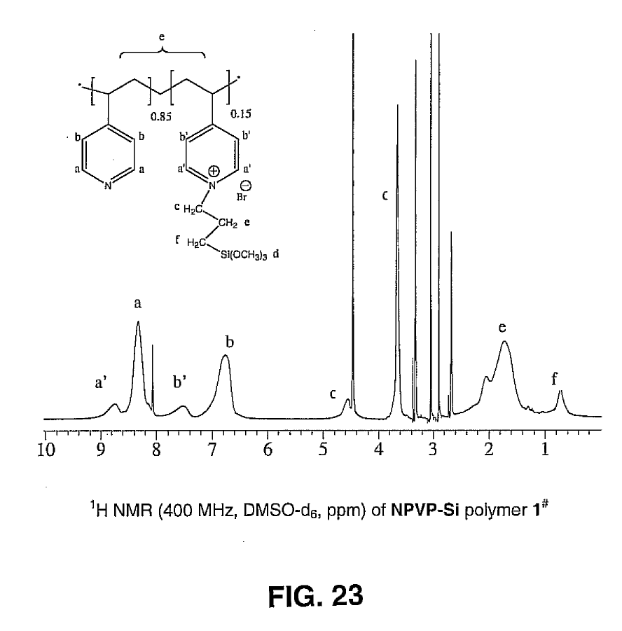

FIG. 23 shows an ¹H NMR spectrum of NPVP—Si polymer 1# (Table 1) with peak assignments shown. The amount of the silane (N-alkylation) was established by comparing peak integration between the pyridine protons a & b, and the —OCH₃ protons labeled d. The ¹³C NMR of polymer 1# (75 MHz, DMSO-d6, ppm) showed peaks at: 162.3, 158.1 147.8, 143.9, 127.0, 124.1, 58.9, 51.3, 48.3, 41.1, 30.9, 9.1.

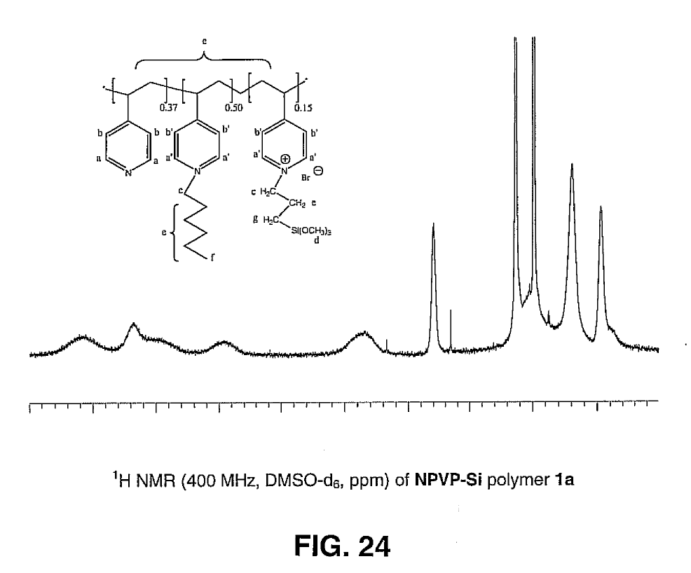

FIG. 24 shows a ¹H NMR spectrum of NPVP—Si polymer 1b (Table 1) with peak assignments shown. The amount of the N-hexylation was established by comparing peak integration of the pyridinium protons a′, and the —OCH₃ protons labeled d. The ¹³C NMR of polymer 1a (75 MHz, DMSO-d6, ppm) showed peaks at: 163.4, 157.0 147.2, 142.2, 127.5, 123.6, 60.8, 57.1, 51.3, 48.3, 40.3, 31.7, 25.9, 22.4, 13.8, 8.3. As characterized from ¹H NMR, the NPVP—Si polymers had around 10-15% of the reactive methoxysilane groups on the pyridinium side chains.

Applications

Composites according to the present invention may be used in applications other than antibacterial films, such as polymer-semiconductor nanoparticle films for displays and other electronic applications, conducting polymers and polymer electrolytes, ink-jet printing materials, catalysts, reaction substrates, and the like. Applications of particle/polymer composites include optical materials, catalysts, and the like.

Antimicrobial modification of surfaces to prevent growth of detrimental microorganisms is useful in various applications. Antibacterial surface coatings according to embodiments of the present invention may be used for hospital surfaces, medical use, textiles, medical implants, and medical devices, surgical instruments, and the like. Antibacterial coatings according to embodiments of the present invention can be used in medical environments, other health industry applications, personal hygiene, food handling and preparation, and any other location where bacterial growth inhibition is desirable. Examples include biomedical devices like catheters, prosthetics, implants, food preparation surfaces, doorhandles and other surfaces touched by multiple persons, and other devices. A composite may be used as a surface coating on a medical implant.

Surface induced contaminations are implicated in food spoilage, spread of food-borne diseases and bio-fouling of materials. Hence, antimicrobial surface coatings can also be used to reduce food poisoning, food borne diseases, skin infections, and the like arising from contact with infected surfaces, for example, using coatings on food preparation or handling materials.

Applications further include water purification. Water may be stored in containers having biocidal composite coatings, or flow through pipes having such coatings on the interior surfaces.

Applications further include improved wound dressings, for example using textiles or hydrogels having antibiotic and antifungal properties. Other applications include drug-eluting materials. Objects having biocidal properties may be formed from composite materials, without use of a substrate.

Further Discussion of Textiles having Antibiotic and Anti-Fungal Properties

Composites of silane group containing polymer and biocide (such as AgBr) particles allow long lasting antibacterial coatings to be formed on commercially available textiles. For example, novel pyridinium-methoxysilane polymers form strong Si—O—Si links to oxide surfaces, thereby anchoring the polymer chains at multiple points and greatly increasing the durability of a coating to a substrate surface. In addition, inter-chain cross-linking of the methoxysilane groups provides additional durability to the coating and makes the coatings highly resistant to solvents and detergent washings. The polymers are soluble in low boiling solvents, and can be easily applied as coats to various textiles via a gas phase aerosol process using commercial paint sprayers, or simple dip coating procedure. In conventional silver treated textiles, the silver can precipitate when exposed to chloride, which is commonly used in fabric cleaning processes. Silver salt containing composites prepared according to embodiments of the present invention were substantially unaffected by bleach, and are highly stable.

Textiles may be treated after manufacture, or may be made using treated fibers. A polymer or polymer composite coating may be applied by dip coating, spraying, or other process. The polymer or composite may be applied in liquid form, for example as a polymer solution, or colloid of particles and polymer.

Injuries caused by impact, such as ballistic projectiles, often lead to non-sterile pieces of textile fragments being embedded inside the resulting wounds. This may cause systemic infections in the recuperating person, and in some cases leads to permanent organ damage, amputations, or death. Antimicrobial textiles according to embodiments of the present invention can significantly reduce wound infections caused by contaminated pieces of clothing.

Alkoxysilane groups within antibacterial cationic polymers may be used to covalently anchor the polymers to textile surfaces to generate long lasting non-leaching antibacterial coatings. Cotton fabric consists of polymeric polysaccharide chains which have a large number of free hydroxyl functionalities capable of reacting with methoxysilane groups.

After an impact injury, embedded pieces of clothing are usually numerous, small, and cannot be removed easily as they are transparent to X-rays. A single component polycationic antimicrobial coating on textiles may be quickly rendered ineffective due to rapid adsorption of negatively charged biomacromolecules (such as proteins, DNA, RNA, polysaccharides), and blood cells like platelets on the surface of the positively charged textile material. Bacterial biofilms have been implicated in a wide variety of lethal outcomes from this and similar situations. A leachable biocidal species such as silver ions can diffuse through the overlying biofilm to kill pathogenic microbes in the surrounding wound environment. Hence, the dual component antibacterial formulations consisting of a non-leaching contact active polymer coating and an embedded leachable AgBr biocide can be extremely effective in reducing infections caused by the insertion of textile materials from traumatic injuries. The slow release of the biocide from the embedded textile fragments in the wound provides an immediate and localized antiseptic action, reducing the incidence of future infection.

Composites including metal salt particles (such as silver salt particles) are typically stable against chloride, a common component of detergents, and other detergent components. Further, the biocidal properties of materials according to embodiments of the present invention, such as silver salt particles, may be substantially unaffected by pH. Hence, silver halide/polymer composites allow biocidal coatings to be formed on textile substrates that are stable against typical textile laundering processes. The polymer component of the composite may be cross-linked, for example by silicon-containing groups such as silane, to further stabilize a composite coating.

Further, bromide-including materials can provide fire retardant properties, so that polymer composites including silver bromide particles may further provide flame retardant properties. Hence, such composites may impart flame retardant properties as well as biocidal properties to textile substrates. Particles

Particles may be nanoparticles e.g. (e.g. 0.5-1000 nm diameter), microparticles (e.g. 500 nm-1 mm diameter), depending on the application. Particles may comprise metal (elemental metal or alloy), oxides, halides, nitrides, sulfides, or other ionic compounds. Using a plurality of reagents, different species of particles may be formed.

In other examples, particles may be formed separately and suspended in a polymer solution, the particles and polymer being applied to the surface using a fluid medium, such as a solution. The lifetime of the biocidal properties can be extended by including larger particle sizes. A particle size distribution may be used to obtain desired silver ion release properties.

### OTHER EXAMPLES

Other examples include composites may comprise any contact active amphiphilic polymers or peptide mimics, which kill bacteria by cell membrane disruption, microbe repelling anti-adhesive polymer, which prevent cell/protein adhesion, and/or other polymer. Polymer or composite materials may be loaded with slow releasing biocides such as metals and metal compounds (such as silver compounds), antibiotics, small molecule biocides, oxidizing anions, halogens and halides, and nitric oxide. Where appropriate, these may be instead of or augmenting biocidal silver ion release.

Patents, patent applications, or publications mentioned in this specification are incorporated herein by reference to the same extent as if each individual document was specifically and individually indicated to be incorporated by reference.

The invention is not restricted to the illustrative examples described above. Examples are not intended as limitations on the scope of the invention. Methods, apparatus, compositions, and the lice described herein are exemplary and not intended as limitations on the scope of the invention. Changes therein and other uses will occur to those skilled in the art. The scope of the invention is defined by the scope of the claims.

Having described our invention, we claim:

1. A method of forming a composite material, the method comprising:
providing a polymer, the polymer including a charged species and a counter ion;
providing a reagent, the reagent including a metal compound;
treating the polymer with the reagent so that the metal compound reacts with the counter ion to form particles of a metal salt,
the particles of the metal salt being distributed through the polymer so as to form the composite material.

2. The method of claim 1, wherein the polymer is provided as a polymer solution or suspension, the method further comprising forming a coating of the composite material on a substrate.

3. The method of claim 1, wherein the particles comprise a biocidal agent, the method being a method of preparing a biocidal composite material.

4. The method of claim 3, wherein the metal compound is a silver compound and the metal salt is a silver salt,
the silver compound reacting with the counter ion to form the silver salt.

5. The method of claim 4, wherein the silver salt is a silver halide.

6. The method of claim 1, wherein the charged species is a nitrogen atom.

7. The method of claim 1, wherein the polymer includes N-alkylated pyridinium groups.

8. The method of claim 1, wherein the polymer is a partially N-substituted poly(4-vinyl pyridine).

9. The method of claim 1, wherein the polymer includes N-alkylated polyallylamine groups.

10. A biocidal material, comprising:
a polymeric matrix, the polymer matrix including a polymer, the polymer being an ionic polymer; and
particles dispersed through the polymer matrix, the particles including a biocidal agent.

11. The biocidal material of claim 10, wherein the ionic polymer is a cationic polymer.

12. The biocidal material of claim 10, wherein the particles include a silver salt, the biocidal agent being silver ions.

13. The biocidal material of claim 12, wherein the silver salt is silver bromide.

14. The biocidal material of claim 10, the particles having a mean diameter between 1 nanometer and 10 microns.

15. The biocidal material of claim 10, the particles having a mean diameter between 1 nanometer and 100 nanometers.

16. The biocidal material of claim 10, wherein the polymer includes a ternary or quaternary nitrogen atom.

17. The biocidal material of claim 10, wherein the polymer is a derivative of poly(4-vinyl pyridine).

18. The biocidal coating of claim 17, wherein the polymer is a copolymer of poly(4-vinyl pyridine) and an N-substituted poly(4-vinyl pyridine).

19. The biocidal material of claim 17, wherein the polymer includes N-alkylated pyridinium groups.

20. The biocidal material of claim 17, wherein the polymer further comprises fluoroalkyl groups.

21. The biocidal material of claim 17, wherein the polymer is a copolymer of an N-alkyl substituted poly(4-vinyl pyridine).

22. The biocidal material of claim 10, the biocidal material being a biocidal coating on a substrate.

23. The biocidal material of claim 22, wherein the polymer includes silicon-containing groups, the polymer being at least partially cross-linked through the silicon-containing groups.

24. The biocidal material of claim 22, wherein the biocidal material is covalently linked to the substrate through silicon-containing groups.

24. The biocidal material of claim 22, wherein the substrate comprises a glass, metal, plastic, ceramic, wood, cellulose, fabric, paper, or an oxide.

26. The biocidal material of claim 22, wherein the substrate is a textile material.

27. The biocidal material of claim 26, wherein the textile material includes fibers comprising fiber material selected from a group of fiber materials consisting of acrylic polymers, acrylate polymers, aramid polymers, cellulosic materials, cotton, nylon, polyolefins, polyester, polyamide, polypropylene, rayon, wool, spandex, silk, and viscose.

28. The biocidal material of claim 26, wherein the particles comprise a silver salt.

29. The biocidal material of claim 26, wherein the silver salt is silver bromide.

30. The biocidal material of claim 26, wherein the polymer is a cationic polymer.

31. The biocidal material of claim 26, wherein the polymer comprises alkylpyridinium groups.

32. The biocidal material of claim 26, wherein the polymer is at least partially cross-linked through silicon-containing groups.

33. A biocidal material, comprising:
a polymer, the polymer being a cross-linked ionic polymer; and
a biocidal counter-ion associated with the ionic polymer.

34. The biocidal material of claim 33, wherein the polymer is a cationic polymer cross-linked through silicon-containing groups.

35. The biocidal material of claim 34, wherein the silicon-containing groups are linked to a charged nitrogen atom through a linking group.

36. The biocidal material of claim 35, wherein the linking group is an alkyl group, the alkyl group being attached to the charged nitrogen atom of a pyridinium group.

37. The biocidal material of claim 33, wherein the biocidal ion is selected from a group consisting of iodide, triiodide, and hypochlorite.
